(19) United States
(12) Patent Application Publication
(10) Pub. No.: US 2002/0151734 A1
(43) Pub. Date: Oct. 17, 2002

(54) 12-HETrE analogs and methods of use thereof

(76) Inventors: Michal Laniado Schwartzman, Pleasantville, NY (US); John R. Falck, University Park, TX (US)

Correspondence Address: Chantal Morgan D'Apuzzo, Ph.D., Wolf, Greenfield & Sacks, P.C., 600 Atlantic Avenue, Boston, MA, 02210, US

(21) Appl. No.: 10/038,763

(22) Filed: Jan. 2, 2002

Related U.S. Application Data
(60) Provisional application No. 60/258,806, filed on Jan. 2, 2001.

(51) Int. Cl. C07C 229/24

(52) U.S. Cl. 554/103

(57) ABSTRACT

The present invention to 12-HETrE analogs which are agonists and antagonists of 12-HETrE. The compositions may be formulated in pharmaceutically acceptable formulations. The invention also includes methods and products for treating inflammatory conditions, neovascularization, tumor growth, cancer, ischemic cardiovascular diseases, and ocular conditions.

### RELATED APPLICATIONS

This application claims priority to U.S. Provisional Application Serial No. 60/258,806, filed Jan. 2, 2001, under 35 U.S.C. §119, the entire contents of which are incorporated herein by reference.

### GOVERNMENT SUPPORT

The research performed in support of the invention may have been supported in part by National Institute of Health Eye Institute EY05613 (41-748). The government may have certain rights in this invention.

### FIELD OF THE INVENTION

The present invention relates to 12-HETrE analogs which are agonists and antagonists of 12-HETrE. The invention also relates to methods and products for treating inflammatory conditions, neovascularization, tumor growth, and ischemic cardiovascular diseases.

### BACKGROUND OF THE INVENTION

12(R)-hydroxy-5,8,14-eicosatrienoic acid (12(R)-HETrE) is an endogenous eicosanoid produced by the metabolism of arachidonic acid. The synthesis of 12(R)-HETrE is increased in inflamed corneal and endothelial tissues. 12(R)-HETrE possesses in vitro and in vivo biological activity indicative of a pro-inflammatory factor.

### SUMMARY OF INVENTION

12-HETrE (12(R)-hydroxy-5,8,14-eicosatrienoic acid) is an endogenous eicosatrienoid whose synthesis is increased in inflamed tissues.

The present invention relates to novel analogs of 12-HETrE. The 12-HETrE analogs include 12-HETrE antagonists which inhibit the activity of 12-HETrE and 12-HETrE agonists, which enhance 12-HETrE activity or have similar activity to 12-HETrE.

According to one aspect of the invention, compositions are provided. These compositions are of the general formula:

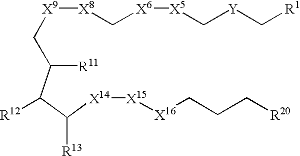

wherein X⁵, X⁶, X⁸, X⁹, X¹⁴, X¹⁵, and X¹⁶ are independently selected from —CH₂—, —CH—, and —C—, and wherein X⁵═X⁶, X⁸═X⁹, and X¹⁴═X¹⁵ or X¹⁵═X¹⁶;

R¹ is selected from —COOH, —CH₃, and —C(O)NHSO₂Z, wherein Z is selected from the group consisting of methyl, paraiodobenzene, —COOH, parabenzylamine, propylamine, and COOR²¹, and wherein R²¹ is alkyl having from 1 to 6 carbons; R¹¹, R¹², and R¹³ are independently selected from —H, —OH, and —OCH₃; R²⁰ is —CH₃ or —COOH; and Y is selected from —CH₂—, —O—, —N—, and —S—.

In some embodiments, only one of R¹¹, R¹², and R¹³ is hydroxy. In other embodiments, at least one of R¹¹, R¹², and R¹³ define a stereocenter with an (R) configuration. In other embodiments, at least one of R¹¹, R¹², and R¹³ define a stereocenter with an (S) configuration. In some embodiments, 12-HETrE agonists have the following formula with three double bonds:

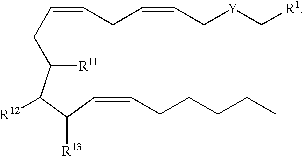

In other embodiments, 12-HETrE agonists and antagonists have the following formula:

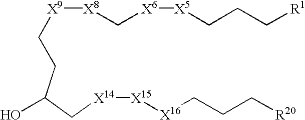

wherein the variable positions are as defined above.

In still other embodiments, 12-HETrE analogs have the following formulas:

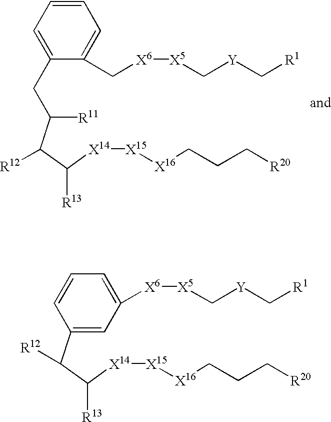

wherein Y, X⁵, X⁶, X⁸, X⁹, X¹⁴, X¹⁵, and X¹⁶ are independently selected from —CH₂—, —CH—, —C—, —N—, —O—, and —S—; R¹ is selected from —COOH, —CH₃, and —C(O)NHSO₂Z, wherein Z is selected from the group consisting of methyl, paraiodobenzene, —COOH, parabenzylamine, propylamine, and COOR²¹, and wherein R²¹ is alkyl having from 1 to 6 carbons; and R¹¹, R¹², and R¹³ are independently selected from of —H, —OH, and —OCH₃; ²⁰ is —CH₃ and —COOH.

Preferred embodiments of 12-HETrE analogs include:

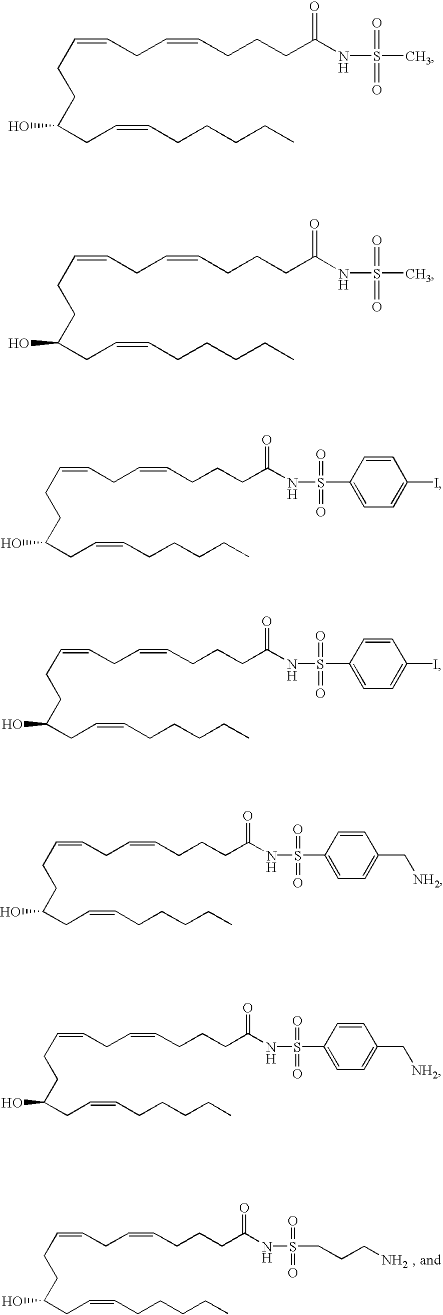

Other preferred embodiments include compounds having the formula:

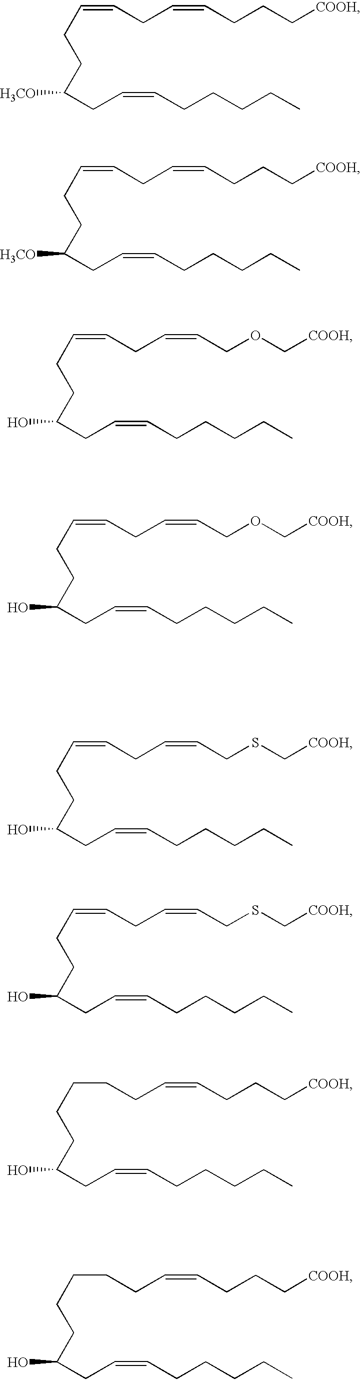

In some embodiments, R¹ is —COOH and R²⁰ is —H. In other embodiments, Y is —CH₂—.

According to another aspect of the invention, methods are provided to synthesize 12-HETrE analogs.

The invention also encompasses methods of treatment using 12-HETrE analogs. According to one aspect of the invention, 12-HETrE antagonists may be administered to a subject for treating or preventing an adverse medical condition characterized by inflammation. According to particular embodiments, the inflammation is characteristic of or results from a skin inflammatory condition, including hypersensitization, and psoriasis, or a corneal inflammatory condition. In adverse medical conditions characterized by inflammation, the inflammation may be mediated by neutrophils, leukocytes, T cells, or a combination. The inflammation may result in vasodilation, an increase in membrane permeability, early neutrophil chemotaxis, late angiogenesis, and so forth.

According to another aspect of the invention, 12-HETrE antagonists may be administered to a subject to treat ocular conditions. In one aspect, a 12-HETrE antagonist is administered in an effective amount to treat ocular conditions. According to particular embodiments, the ocular condition is or results from corneal angiogenesis, corneal transplantation, injury due to contact lens wear, trachoma, infectious conditions, retinal neovascularization, choroidial neovascularization, retinopathy, and age-related macular degeneration. According to particular embodiments, methods of treating retinopathy resulting from prematurity or diabetes are provided.

The invention also encompasses methods of treating cardiovascular disorders by administering a 12-HETrE agonist to a subject. In particular embodiments, the cardiovascular disorder is an ischemic condition, which may result from or include stroke, myocardial infarction, coronary artery disease, and chronic exercise. In one embodiment, the 12-HETrE agonist is administered to a subject having an ischemic disease or condition, in conjunction with an antigenic factor.

In yet another aspect of the invention, methods for treating cancer in a subject are provided by administering a 12-HETrE antagonist to the subject. The 12-HETrE analogs of the invention are capable of inhibiting cell growth in tumor cells.

The invention provides a composition of a 12-HETrE analog for use as a medicament. The invention also provides a composition of a 12-HETrE analog for use in the manufacture of a medicament for the treatment or prevention of adverse medical conditions, including adverse medical conditions characterized by inflammation, ocular conditions, and cardiovascular conditions.

In another aspect of the invention, methods for treating cancer using 12-HETrE analogs are provided. In one embodiment, a 12-HETrE antagonist is administered in an effective amount to inhibit tumor growth in a subject.

Each of the limitations of the invention can encompass various embodiments of the invention. Therefore, it is anticipated that each of the limitations of the invention involving any one element or combinations of elements can be included in each method and product.

Other aspects, features and advantages of the present invention will become apparent from the following Detailed Description and Examples. It should be understood, however, that the detailed description and the specific examples, while indicating many embodiments of the invention, are given by way of illustration only, since various changes and modifications within the spirit and scope of the invention will become apparent to those skilled in the art from this Detailed Description.

### DETAILED DESCRIPTION

The invention involves the discovery that certain 12-HETrE analogs function as antagonists of 12-HETrE and others function as agonists of 12-HETrE.

There are two isomers of 12-hydroxy-5,8,14-eicosatrienoic acid (12-HETrE). 12(R) hydroxy-HETrE has the formula:

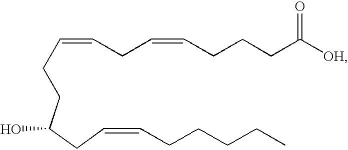

and 12(S) hydroxy-HETrE has the formula:

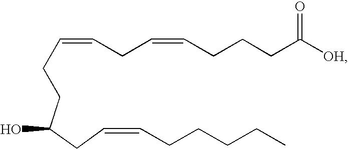

In some embodiments, the 12-HETrE analogs of the invention have the following general formula:

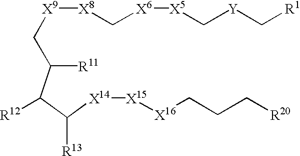

wherein X⁵, X⁶, X⁸, X⁹, X¹⁴, X¹⁵, and X¹⁶ are independently selected from —CH₂—, —CH—, and —C—, and wherein X⁵═X⁶, X⁸═X⁹, and X¹⁴═X¹⁵ or X¹⁵═X¹⁶; R¹ is selected from —COOH, —CH₃, and —C(O)NHSO₂Z, wherein Z is selected from methyl, paraiodobenzene, —COOH, parabenzylamine, propylamine, and COOR²¹, and wherein R²¹ is alkyl having from 1 to 6 carbons; R¹¹, R¹², and R¹³ are independently selected from —H, —OH, and —OCH₃; R²⁰ is —CH₃ or —COOH; and Y is selected from —CH₂—, —O—, —N—, and —S—. However, compounds wherein X⁵, X⁸, and X¹⁴ are —CH—, Y is —CH₂—, R¹ is —COOH, R¹¹ and R¹³ are both H, R²⁰ is —CH₃, and R¹² is OH are not encompassed by the invention. In other words, the invention does not encompass 12-hydroxy-5,8,14-eicosatrienoic acid (12-HETrE).

As used herein, the definitions of all substituents is constant, such that R¹, R¹¹, X⁵, X⁸, etc. maintain a constant meaning throughout, even with reference to different 12-HETrE analogs. Generally, embodiments described for one general formula are applicable for other general formulas as well.

In some embodiments, at least one of R¹¹, R¹², and R¹³, define a stereocenter with an (R) configuration. In other embodiments, at least one R¹¹, R¹², and R¹³ define a stereocenter with an (S) configuration. In preferred embodiments, only one of R¹¹, R¹², and R¹³ is not a hydrogen. In preferred embodiments, one of R¹¹, R¹², and R¹³ is —OH, and two are —H.

In some embodiments, the 12-HETrE analogs have three double bonds, for example, when X⁵, X⁸, and X¹⁴ are each —CH—. In other embodiments, the 12-HETrE analogs have two double bonds and X⁵ is —CH₂— and X⁸ and X¹⁴ are each —CH—. In other embodiments, R¹ is —COOH and R²⁰ is H. In another preferred embodiment, Y is —CH₂—.

In some embodiments, the 12-HETrE analogs have the following formula:

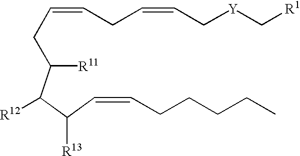

wherein R¹, R¹¹, R¹², R¹³, and Y are as described above.

In some embodiments, R¹ is —C(O)NHSO₂Z wherein Z is methyl, paraiodobenzene, parabenzylamine, propylamine carboxylic acid (—COOH), and an ester (—COOR²¹), wherein R²¹ is alkyl having from 1 to 6 carbons. Preferred embodiments include:

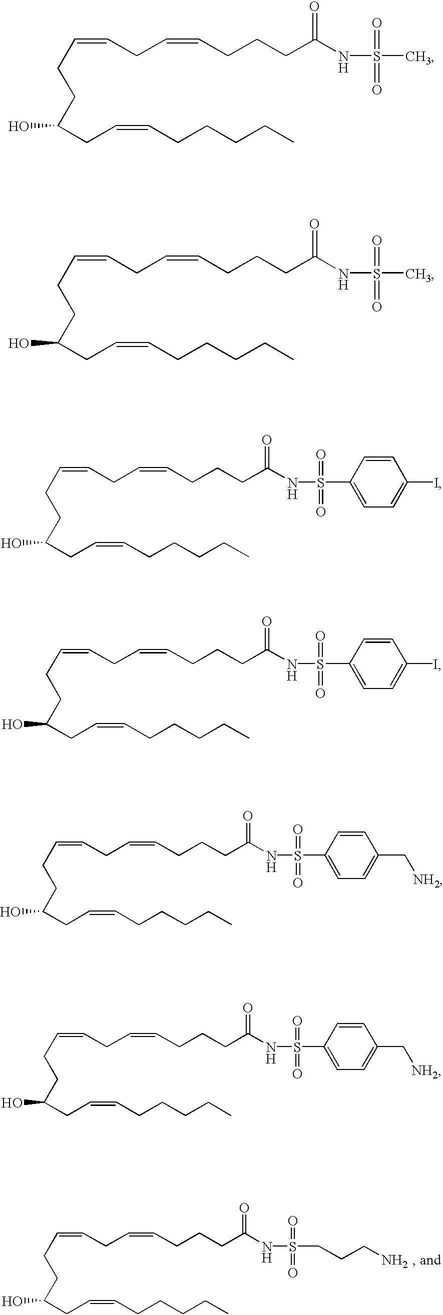

In some embodiments, R¹ is —COOH, and R²⁰ is H. Preferred compounds include:

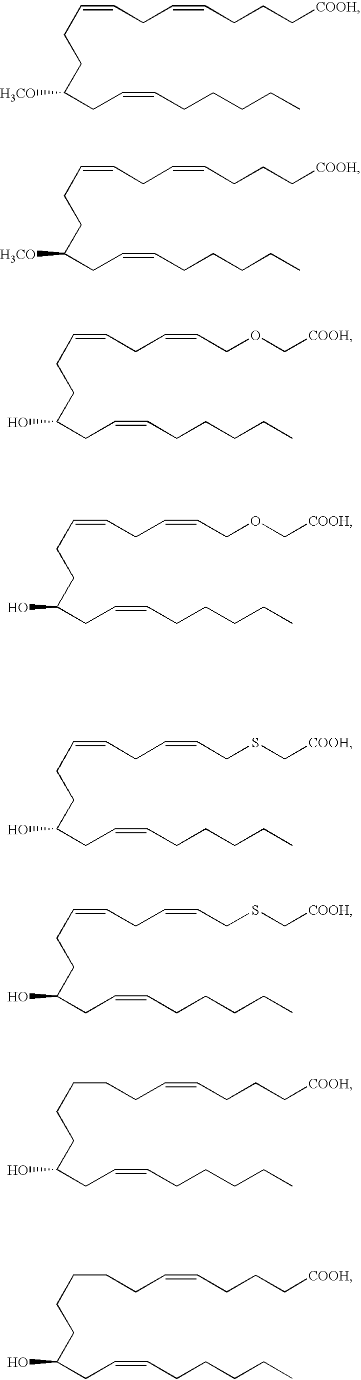

In other preferred embodiments, R¹¹ and R¹³ are each —H and R¹² is —OH, Y is —CH₂—. Preferred embodiments include:

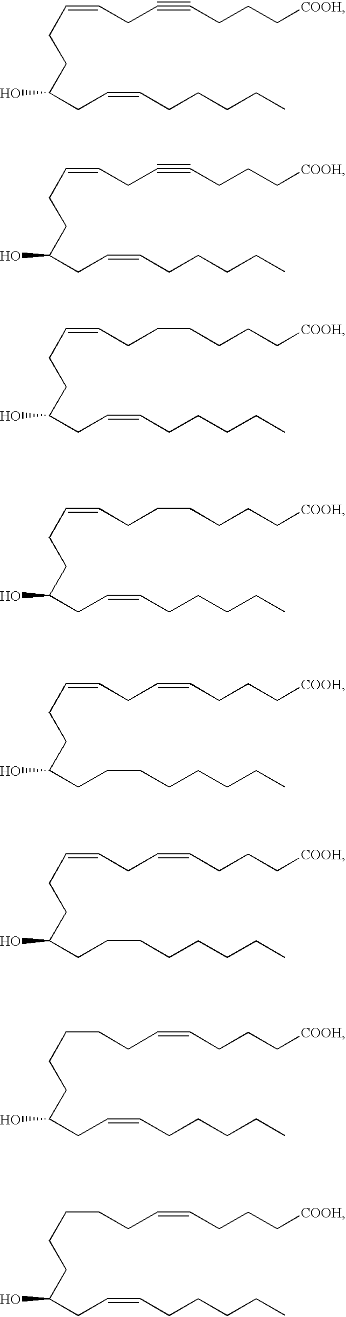

In still other embodiments, aromatic rings may be incorporated into the fatty acid carbon chain. Such compounds include 12-HETrE analogs of the formulas:

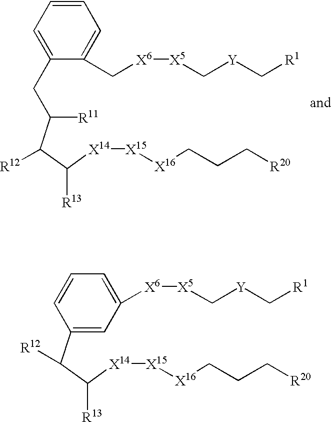

According to another aspect of the invention, 12-HETrE analogs encompass analogs having heteroatoms substituted in the chain. “Heteroatom,” as used herein, includes nitrogen, oxygen, and sulfur. Thus, the invention encompasses molecules of the formula:

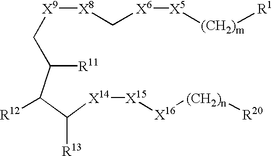

12-HETrE analogs include both agonists and antagonists. As used herein, a 12-HETrE agonist is a molecule encompassed by the above 12-HETrE analog formulas that are structurally similar to but may have a different biological activity from 12-HETrE. An is effective amount of a 12-HETrE agonist for treating cardiovascular disorders in a subject can be easily assessed by any method known in the art.

As used herein, a “12-HETrE antagonist” is a molecule encompassed by the above formulas that inhibit 12-HETrE activity. An effective amount of a 12-HETrE antagonist for treating or preventing inflammation in a subject or for treating an ocular condition in a subject can be determined using any method known in the art.

The 12-HETrE analogs of the invention may be synthesized using methods that will be understood by those skilled in the art. Exemplary synthetic schemes outlined below, and described in more detail in the Examples.

In one embodiment, the invention provides the 12-HETrE analog, N-(methylsulfonyl-12(R)-hydroxyeicosa-5(Z), 8(Z),14(Z)-trienamide (1-a-(R)). Compound 1-a-(R) can be made following Scheme 1 below which is described in more detail in Example 1.

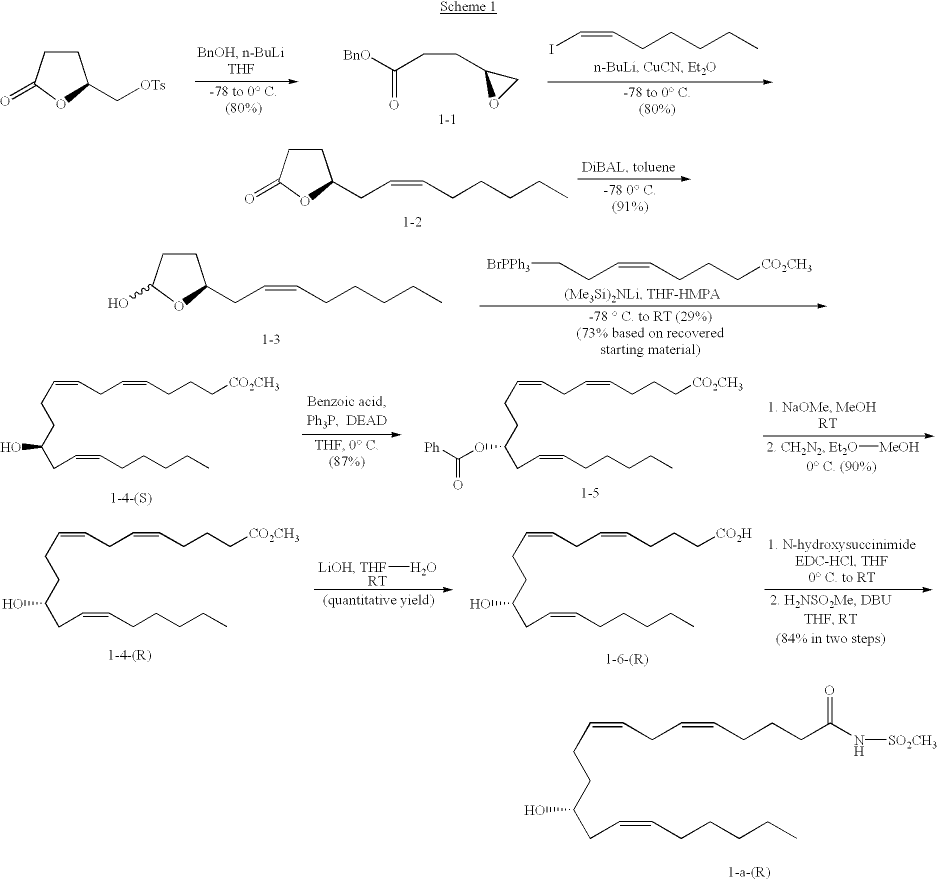

In another embodiment, the 12-HETrE analog 12(R)-Methoxyeicosa-5(Z),8(Z),14(Z)-trienoic acid (2-a-(R)) is provided. Compound 2-a-(R) can be synthesized by using the methodology outlined in Scheme 2 and described in Example 3.

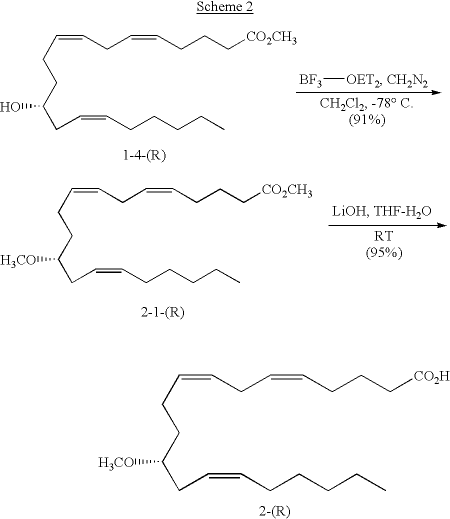

In another embodiment, the 12-HETrE analog 12(R)-hydroxyeicosa-8(Z),14(Z)-dien-5-ynoic acid (3-(R)) is provided. Compound 3-(R) can be synthesized as outlined in Scheme 3 and as described in Example 5.

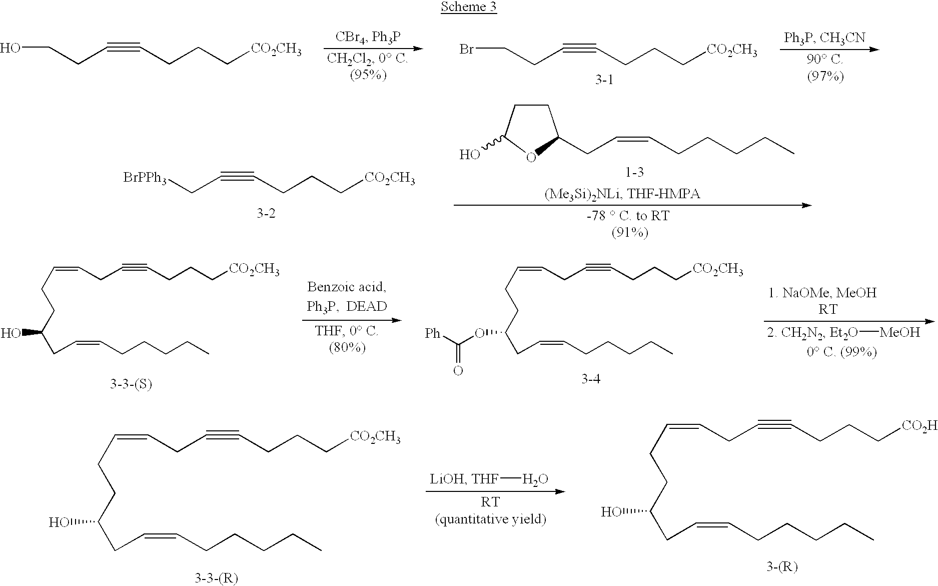

In yet another embodiment, 12-HETrE analog (9(R)-hydroxyheptadeca-2(Z),5(Z),11(Z)-trienyloxy)acetic acid (4-a-(R)) is provided. Compound 4-a-(R) can be synthesized as outlined in Scheme 4 below and described more fully in Example 7.

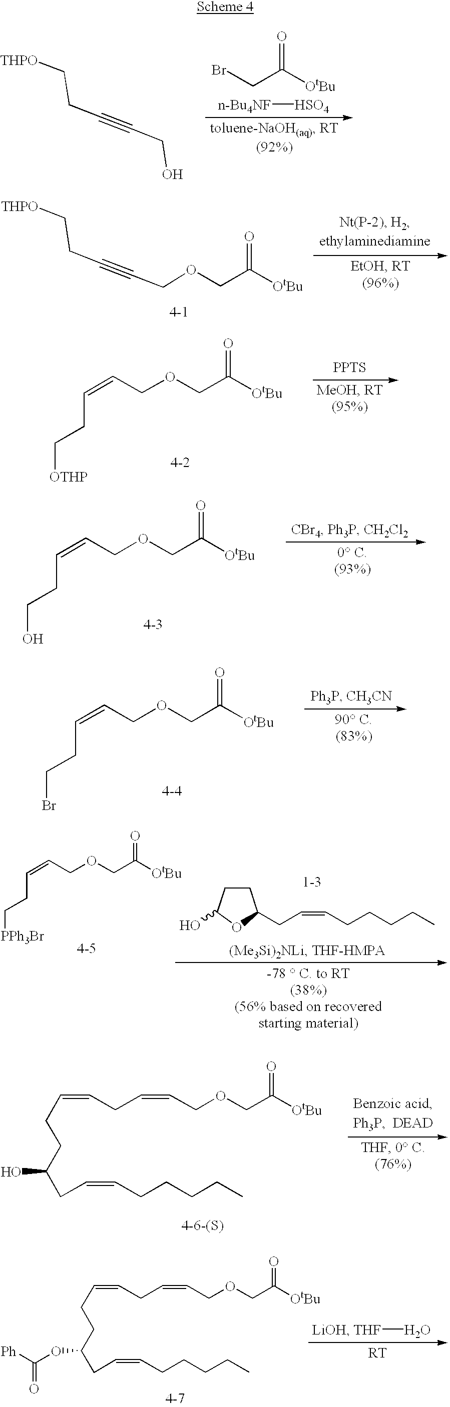

In another embodiment, the 12-HETrE analog 12(R)-hydroxyeicosa-8(Z),14(Z)-dienoic acid (5-(R)) is provided. Compound 5-(R) can be synthesized as outlined in Scheme 5 below and as described in Example 9.

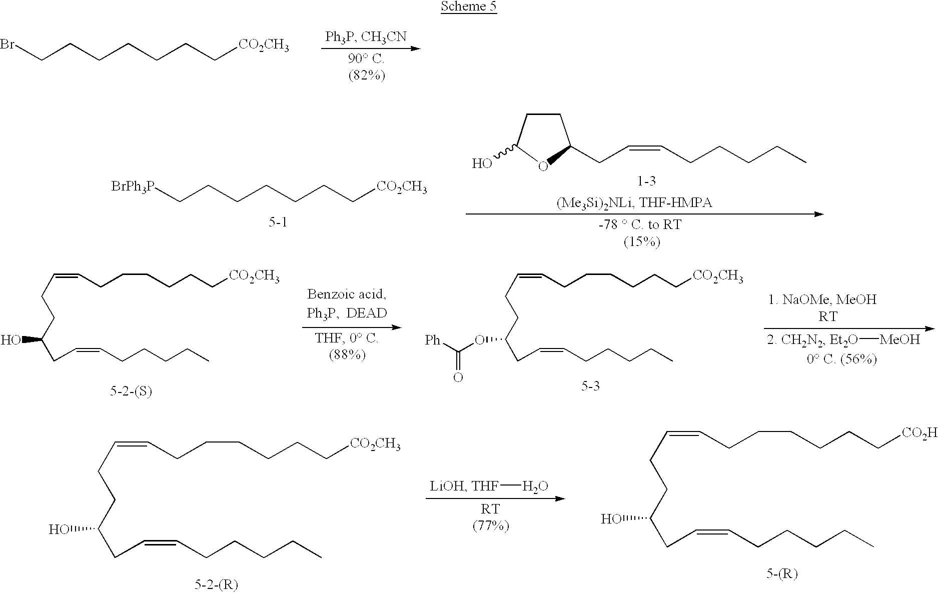

In still another embodiment, the 12-HETrE analog 12(S)-hydroxyeicosa-5(Z),8(Z)-dienoic acid (6-(S)) is provided. Compound 6-(S) can be synthesized as outlined in Scheme 6 and as described in Example 11.

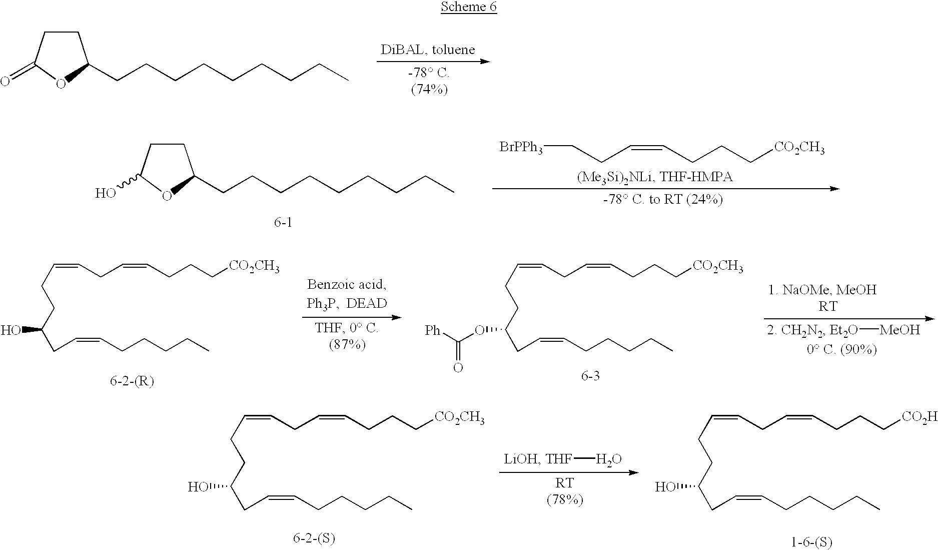

In still another embodiment of the invention, the 12-HETrE analog 12(R)-hydroxy-eicosa-5(Z),14(Z)-dienoic acid (7-(R)) is provided. Compound 7-(R) can be synthesized as outlined in Scheme 7 below and as described more fully in Example 13.

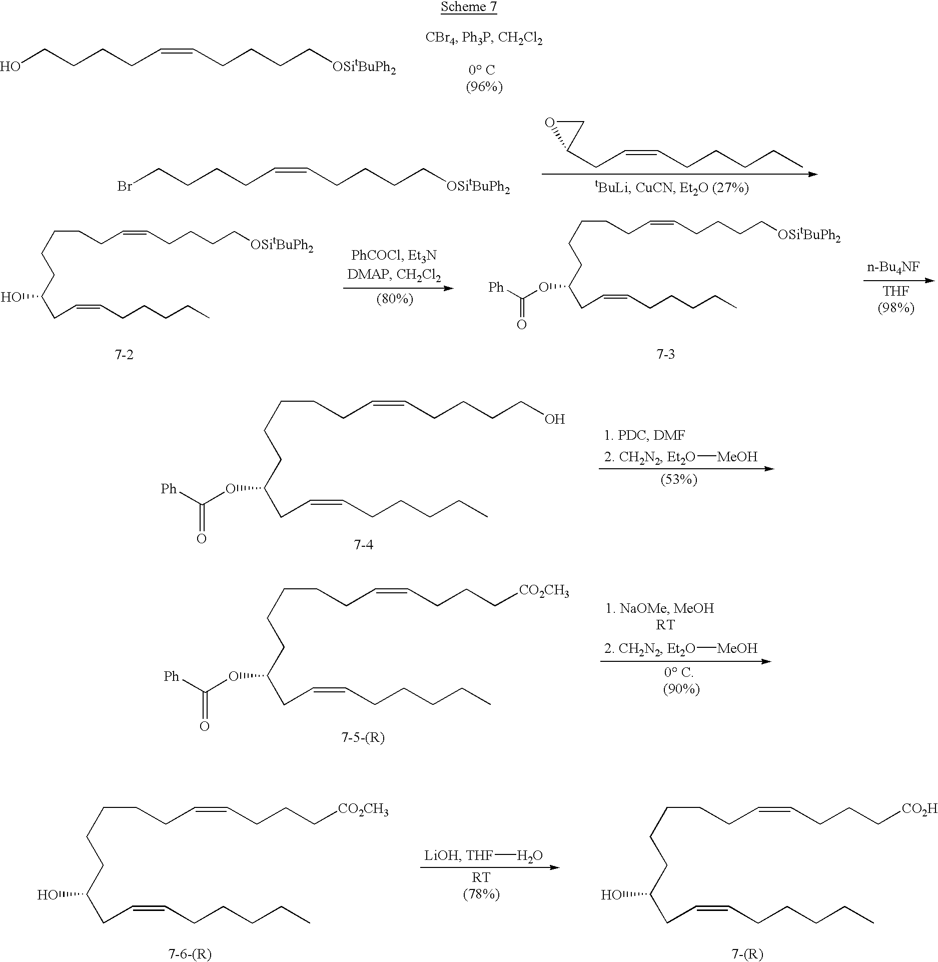

It will be readily apparent to those skilled in the art to generally use the methods described in the synthetic schemes and examples to produce other 12-HETrE analogs of the invention.

The invention includes compositions as well as methods for treating a subject having an adverse medical condition by administering a 12-HETrE agonist or antagonist to a subject. In one embodiment, a subject having an adverse medical condition is one who has an ocular condition. In an other embodiment, a subject having an adverse medical condition is one who has a cardiovascular disorder. In another embodiment, a subject having an adverse medical condition characterized by inflammation is one who has an inflammatory disease or is at risk of developing an inflammatory disease. In yet another embodiment, a subject may be administered a 12-HETrE analog to treat cancer.

As used herein, “a subject” includes humans, non-human primates, dogs, cats, horses, sheep, goats, cows, rabbits, pigs, and rodents. As used herein, “inflammatory disease” or “inflammatory condition” refers to any condition characterized by local inflammation at a site of injury or infection and includes autoimmune diseases, skin inflammatory conditions, corneal inflammatory conditions, certain forms of infectious inflammatory states, undesirable neutrophil activity characteristic of organ transplants or other implants. These conditions include, but are not limited to hypersensitization, psoriasis, meningitis, cerebral edema, arthritis, nephritis, adult respiratory distress syndrome, pancreatitis, myositis, neuritis, connective tissue diseases, phlebitis, arteritis, vasculitis, allergy, anaphylaxis, ehrlichiosis, gout, organ transplants and/or ulcerative colitis.

An “ocular condition,” as used herein, refers to a condition characterized by ocular surface inflammation. These conditions include, but are not limited to, corneal angiogenesis, corneal transplantation, injury due to contact lens wear, trachoma, infectious conditions, retinal neovascularization, choroidal neovascularization, retinopathy, and age-related macular degeneration. The ocular condition of retinopathy includes, but is not limited to, retinopathy of prematurity and diabetic retinopathy. Ocular conditions are often caused by stimuli such as infections, surgical trauma, and hypoxia, which may be caused by eyelid closure and prolonged contact lens wear.

A “cardiovascular disorder,” as used herein, refers to a number of disorders in the heart and vascular system. Cardiovascular disorders include, but are not limited to, hypertension, angina, cardiac arrhythmias, and ischemic conditions.

Cardiovascular disorders can be treated using either 12-HETrE agonists or antagonists, but generally 12-HETrE agonists are preferred.

An “ischemic condition,” as used herein, refers to a condition characterized by local inflammation resulting from an interruption in the blood supply to a tissue due to a blockage or hemorrhage of the blood vessel responsible for supplying blood to the tissue. Ischemic conditions include, but are not limited to, stroke, myocardial infarction, coronary artery disease, chronic exercise, and cerebral ischemia.

As used herein, cardiovascular disorders also include angiogenesis-mediated diseases which include, but are not limited to, solid tumors, blood born tumors such as leukemia's, tumor metastasis, benign tumors, for example hemangiomas, acoustic neuromas, neurofibromas, trachomas, and pyogenic granulomas, pre-malignant tumors, rheumatoid arthritis, psoriasis, ocular angiogenic diseases, for example, diabetic retinopathy, retinopathy of prematurity, macular degeneration, corneal graft rejection, neovascular glaucoma, retrolental fibroplasia, rubeosis, Osler-Webber Syndrome, myocardial angiogenesis, plaque neovascularization; telangiectasia, hemophiliac joints, angiofibroma, and wound granulation.

In one aspect of the invention, the 12-HETrE analogs of the invention are useful to treat cancer. 12-HETrE antagonists can be administered to tumor cells to inhibit cell growth. In one embodiment, the 12-HETrE antagonists are co-administered with other anticancer therapeutics.

A “subject having a cancer” is a subject that has detectable cancerous cells. The cancer may be a malignant or non-malignant cancer. Cancers or tumors include but are not limited to biliary tract cancer; brain cancer; breast cancer; cervical cancer; choriocarcinoma; colon cancer; endometrial cancer; esophageal cancer; gastric cancer; intraepithelial neoplasms; lymphomas; liver cancer; lung cancer (e.g. small cell and non-small cell); melanoma; neuroblastomas; oral cancer; ovarian cancer; pancreas cancer; prostate cancer; rectal cancer; sarcomas; skin cancer; testicular cancer; thyroid cancer; and renal cancer, as well as other carcinomas and sarcomas.

A “subject at risk of having a cancer” as used herein is a subject who has a high probability of developing cancer. These subjects include, for instance, subjects having a genetic abnormality, the presence of which has been demonstrated to have a correlative relation to a higher likelihood of developing a cancer and subjects exposed to cancer causing agents such as tobacco, asbestos, or other chemical toxins, or a subject who has previously been treated for cancer and is in apparent remission. When a subject at risk of developing a cancer is treated with the 12-HETrE analogs of the invention, the subject may be able to kill the cancer cells as they develop.

The antagonistic or agonistic activity of 12-HETrE analogs may be measured by measuring increases in vascular cadothelial growth factor (VEGF) mRNA following incubation of microvessel endothelial cells (RLMVE cells) with 12(R)-HETrE and other compounds followed by slot blot analysis, as described in the Examples. 12-HETrE agonists increase VEGF mRNA production and 12-HETrE agonists do not. In this way the 12-HETrE analogs 8-(R) and 8-(S) were determined to be 12(R)-HETrE agonists.

Alternatively, the antagonistic or agonistic activity of 12-HETrE analogs may be measured by determining the percentage of inhibition of [³H]12(R)-HETrE binding at 10 nM to RLME cells. Generally, inhibition of [³H]12(R)-HETrE binding greater than 50% is indicative of agonists and inhibition lower than 50% is indicative of antagonists. Representative data for some of the 12-HETrE analogs described in the Examples is shown in Table 1.

TABLE 1
Percentage inhibition of 12-HETrE analogs (1 μM) on [³H]12(R)-HETrE
binding at 10 nM to RLME cells.

| Compound | % Inhibition | Result |
|---|---|---|
| 12(R)-HETrE | 69.8 | — |
| 12(S)-HETrE | 62.4 | Agonist |
| 1-a-(R) | 62.6 | Agonist |
| 1-a-(S) | 33.3 | Antagonist |
| 3-(R) | 58.1 | Agonist |
| 3-(S) | 23.6 | Antagonist |
| 4-a-(R) | 48.0 | Antagonist |
| 4-a-(S) | 24.3 | Antagonist |
| 2-(R) | 38.6 | Antagonist |
| 2-(S) | 34.2 | Antagonist |
| 7-(R) | 88.7 | Agonist |
| 7-(S) | 69.5 | Agonist |
| 6-(S) | 73.0 | Agonist |
| 6-(R) | 87.2 | Agonist |
| 5-(R) | 92.4 | Agonist |
| 5-(S) | 54.5 | Agonist |

Effective amounts will depend, of course, on the severity of the condition; individual patient parameters including age, physical condition, size and weight; concurrent treatments; frequency of treatment; and the mode of administration. These factors are well known to those of ordinary skill in the art and can be addressed with no more than routine experimentation. It is preferred generally that a maximum dose be used, that is, the highest safe dose according to sound medical judgment.

It is expected that intravenous dosage of the 12-HETrE analogs in the range of 0.1 to 20 mg/kg/min, in one or several administrations, will yield the desired results. A preferred daily dose of a 12-HETrE analog is 1.0 mg/kg/min. In the event that the response in a subject is insufficient at such doses, even higher doses, (or effectively higher doses by a different routes of administration, for example, a more localized delivery route) may be employed to the extent that the patient tolerance permits. Multiple doses per day are contemplated to achieve effective and constant systemic levels of the 12-HETrE analog compound.

The present invention also includes a pharmaceutical composition having a therapeutically effective amount of 12-HETrE analog included in a pharmaceutically acceptable carrier. The term “pharmaceutically acceptable carrier,” as used herein, means one or more compatible solid or liquid fillers, dilutants or encapsulating substances which are suitable for administration to a human or other animal. The term “carrier” denotes an organic or inorganic ingredient, natural or synthetic, with which the active ingredient is combined to facilitate the application. The components of the pharmaceutical compositions also are capable of being commingled with 12-HETrE analog and with each other, in a manner such that there is no interaction which would substantially impair the desired pharmaceutical efficacy.

The pharmaceutical preparation of the invention includes a 12-HETrE analog and a pharmaceutically acceptable carrier. In one embodiment, the 12-HETrE analog is a synthetic preparation of 12-HETrE analog. As used herein “a synthetic preparation of 12-HETrE analog” includes a preparation of 12-HETrE analog that is chemically derived. The chemically derived 12-HETrE analog may be made by any chemical procedure known in the art. Examples of procedures used to synthesize 12-HETrE analogs are provided in Examples 1-14. The compounds useful in the practice of the invention can be prepared in accordance with the reaction described in Examples 1-14 below or through modifications thereof, that will be readily apparent to those skilled in the art. A suitable protocol to synthesize a 12-HETrE analog can be selected with due consideration of the particular substituents, commercial availability of some starting materials, and the like.

As used herein, “12-HETrE analogs” include analogs which may have one or more stereocenters with an (R) configuration and/or an (S) configuration. 12-HETrE analogs also encompass achiral compounds. In other words, 12-HETrE analogs encompass 12(S)-hydroxy-5,8,14-eicosatrienoic acid analogs 12(R)-hydroxy-5,8,14-eicosatrienoic acid analogs, and compounds having hydroxy substituents at any or all of the 11, 12, and 13 positions.

A pharmaceutical preparation of a 12-HETrE analog may be used alone or in combination with a therapeutic agent for treating an inflammatory disease or condition, an ocular condition, or a cardiovascular disorder. Non-therapeutics for treating these diseases and conditions are described in medical text books, such as Harrison's Principals of Internal Medicine (McGraw Hill, Inc., NY). The particular therapeutic agent used depends on the nature of the disease or condition being treated.

Therapeutics useful in the treatment of inflammatory diseases or conditions involving infectious agents include various antipathogen agents, i.e., antibiotics, antivirals, antifungals and antiparasitics. The type and concentration of therapeutic depends inter alia on the infectious agent causing the inflammatory disease or condition. For example, chloramphenicol is therapeutically useful for the treatment of meningitis due to Streptococcus pneumoniae, Haemophilus influenzae, and Neisseria meningitides but not in the treatment of meningitis due to E. Coli or Klebsiella pneumoniae. Cefotaxime, ceftizoxime, ceftriaxone, ceftazidime, and moxalactam are useful in treating all forms of meningitis. Penicillin may also be used to treat S. pneumoniae and N. meningitides.

In general, therapeutics from the group comprising antibiotics include, for example, tetracycline antibiotics, such as chlortetracycline, oxytetracycline, tetracycline, demethylchlortetracycline, metacycline, doxycycline, minocycline and rolitetracycline; aminoglysodes, such as kanamycin, amikacin, gentamicin C₁ₐ, C₂, C_{2b} or C₁, sisomicin, netilmicin, spectinomycin, streptomycin, tobramycin, neomycin B, dibekacin and kanendomycin; macrolides, such as maridomycin and erythromycin; lincomycins, such as clindamycine and lincomycin; penicillanic acid (6-APA)- and cephalosporanic acid (7-ACA)-derivatives having (6β- or 7β-acylamino groups, respectively, which are present in fermentatively, semi-synthetically or totally synthetically obtainable 6β-acylaminopenicillanic acid or 7β-acylaminocephalosporanic acid derivatives and/or 7β-acylaminocephalosporanic acid derivatives that are modified in the 3-position, such as penicillanic acid derivatives that have become known under the names penicillin G or V, such as phenethicillin, propicillin, nafcillin, oxycillin, cloxacillin, dicloxacillin, flucloxacillin, cyclacillin, epicillin, mecillinam, methicillin, azlocillin, sulbenicillin, ticarcillin, mezlocillin, piperacillin, carindacillin, azidocillin or ciclacillin, and cephalosporin derivatives that have become known under the names cefaclor, cefuroxime, cefazlur, cephacetrile, cefazolin, cephalexin, cefadroxil, cephaloglycin, cefoxitin, cephaloridine, cefsulodin, cefotiam, ceftazidine, cefonicid, cefotaxime, cefmenoxime, ceftizoxime, cephalothin, cephradine, cefamandol, cephanone, cephapirin, cefroxadin, cefatrizine, cefazedone, ceftrixon and ceforanid; and other β-lactam antibiotics of the clavam, penem and carbapenen type, such as moxalactam, clavulanic acid, nocardicine A, sulbactam, aztreonam and thienamycin; and antibiotics of the bicozamycin, novobiocin, chloramphenicol or thiamphenicol, rifampicin, fosfomycin, colistin and vancomycin

Anti-virals include Zidovudine (AZT-Retrovir), Zalcitabine (Hivid-ddC), Dicanosine (Videx-ddI), Protease inhibitors of retroviruses, integrase inhibitors of retroviruses and others well known to those skilled in the art. Other therapeutics useful in the treatment of inflammatory diseases or conditions include, but are not limited to, anti-inflammatory agents, or antiphlogistics. Antiphlogistics are, for example, glucocorticoids, such as, cortisone, hydrocortisone, prednisone, prednisolone, fluorcortolone, triamcinolone, methylprednisolone, prednylidene, paramethasone, dexamethasone, betamethasone, beclomethasone, fluprednylidene, desoxymethasone, fluocinolone, flumethasone, diflucortolone, clocortolone, clobetasol and fluocortin butyl ester; immunosuppressive agents; penicillamine; hydroxychloroquine; and nonsteroidal inflammation-inhibitors (NSAID) which encompass anti-inflammatory, analgesic, and antipyretic drugs such as salicyclic acid, difanisal and from the group comprising substituted phenylacetic acid salts or 2phenylpropionic acid salts, such as alclofenac, ibufenac, ibuprofen, clindanac, fenclorac, ketoprofen, fenoprofen, indoprofen, fenclofenac, diclofenac, flurbiprofen, pirprofen, naproxen, benoxaprofen, carprofen and cicloprofen; oxicam derivatives, such as piroxicam; anthranilic acid derivatives, such as mefenamic acid, flufenamic acid, tolfenamic acid and meclofenamic acid; anilino-substituted nicotinic acid derivatives, such as the fenamates miflumic acid, clonixin and flunixin; heteroarylacetic acids wherein heteroaryl is a 2-indol-3-yl or pyrrol-2-yl group, such as indomethacin, oxmetacin, intrazol, acemetazin, cinmetacin, zomepirac, tolmetin, colpirac and tiaprofenic acid; idenylacetic acid of the sulindac type; analgesically active heteroaryloxyacetic acids, such as benzadac; phenylbutazone; etodolac; and nabumetone.

Other therapeutics useful in the treatment of inflammatory diseases or conditions include antioxidants. Antioxidants may be natural or synthetic. Antioxidants are, for example, superoxide dismutase (SUD), 21 aminosteroids/aminochromans, vitamin C or E, etc. Many other antioxidants are well known to those of skill in the art.

The pharmaceutical preparation of the 12-HETrE analog also may be used alone or in combination with a therapeutic agent for treating an ischemic disease or condition. Therapeutics for treating ischemic diseases or conditions are described in medical textbooks such as Harrison's Principles of Internal Medicine (McGraw Hill, Inc., New York ). The particular therapeutic used depends on the nature of the disease or condition. Examples of therapeutics useful in the treatment of ischemic diseases or conditions include anticoagulation agents, antiplatelet agents, and thrombolytic agents.

Anticoagulation agents prevent the coagulation of blood components and thus prevent clot formation. Anticoagulants include, but are not limited to, heparin, warfarin, coumadin, dicumarol, phenprocoumon, acenocoumarol, ethyl biscoumacetate, and indandione derivatives.

Antiplatelet agents inhibit platelet aggregation and are often used to prevent thromboembolic stroke in patients who have experienced a transient ischemic attack or stroke. Antiplatelet agents include, but are not limited to, aspirin, thienopyridine derivatives such as ticlopodine and clopidogrel, dipyridamole and sulfinpyrazone, as well as RGD mimetics and also antithrombin agents such as, but not limited to, hirudin.

For example, the pharmaceutical preparation of 12-HETrE analogs useful to treat cardiovascular disorders may be used in combination with angiogenic factors to treat the cardiovascular disorder. Angiogenic factors promote the growth of new blood vessels. These angiogenic factors include, but are not limited to, angiogenin, angiopoietin-1, Del-1, acidic fibroblast growth factor, basic fibroblast growth factor, follistatin, granulocyte colony-stimulating factor, hepatocyte growth factor, scatter factor, interleukin-8, leptin, midkine, placental growth factor, platelet-derived endothelial cell growth factor, platelet-derived growth factor-BB, pleiotrophin, proliferin, transforming growth factor-alpha, transforming growth factor-beta, tumor necrosis factor-alpha, vascular endothelial growth factor, and vascular permeability factor.

The 12-HETrE analogs may be administered alone or may be delivered in a mixture with other drugs, such as those disclosed above, for treating the inflammatory or ischemic disease or condition. In some embodiments, a common administration vehicle (e.g., pill, tablet, implant, injectable solution, etc.) would contain both the 12-HETrE analog useful in this invention and the therapeutic for treating the inflammatory or ischemic disease or condition, for example. Thus, the present invention also provides pharmaceutical compositions, for medical use, which comprise the 12-HETrE analog of the invention together with one or more pharmaceutically acceptable carriers thereof and optionally other therapeutic ingredients.

In addition to the therapeutic uses of the 12-HETrE analogs, these compounds are also useful for a variety of in vitro purposes. For example, these compounds are useful in competition assays as well as intermediates or starting material for the synthesis of other compounds.

When administered, the formulations of the invention are applied in pharmaceutically acceptable amounts and in pharmaceutically acceptable compositions. Such preparations may routinely contain salts, buffering agents, preservatives, compatible carriers, and optionally other therapeutic ingredients. When used in medicine the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically acceptable salts include, but are not limited to, those prepared from the following acids: hydrochloric, hydrobromic, sulphuric, nitric, phosphoric, maleic, acetic, salicylic, p-toluene sulfonic, tartaric, citric, methane sulfonic, formic, malonic, succinic, naphthalene-2-sulfonic, and benzene sulfonic. Also, pharmaceutically acceptable salts can be prepared as alkaline metal or alkaline earth salts, such as sodium, potassium or calcium salts of the carboxylic acid group.

Suitable buffering agents include: acetic acid and a salt (1-2% W/V); citric acid and a salt (1-3% W/V); boric acid and a salt (0.5-2.5% W/V); and phosphoric acid and a salt (0.8-2% W/V).

Suitable preservatives include benzalkonium chloride (0.003-0.03% W/V); chlorobutanol (0.3-0.9% W/V); parabens (0.01-0.25% W/V) and thimerosal (0.004-0.02% W/V).

Generally, daily oral doses of active compounds will be from about 0.01 milligrams/kg per day to 1000 milligrams/kg per day. It is expected that oral doses in the range of 50 to 500 milligrams/kg, in one or several administrations per day, will yield the desired results. Dosage may be adjusted appropriately to achieve desired drug levels, local or systemic, depending upon the mode of administration. In the event that the response in a subject is insufficient at such doses, even higher doses (or effective higher doses by a different, more localized delivery route) may be employed to the extent that patient tolerance permits. Multiple doses per day are contemplated to achieve appropriate systemic levels of compounds.

A variety of administration routes are available. The particular mode selected will depend, of course, upon the particular combination of drugs selected, the severity of the condition or disorder being treated or prevented, the condition of the patient, and the dosage required for therapeutic efficacy. The methods of this invention, generally speaking, may be practiced using any mode of administration that is medically acceptable, meaning any mode that produces effective levels of the active compounds without causing clinically unacceptable adverse effects. Such modes of administration include oral, rectal, topical, nasal, direct injection, transdermal, sublingual or other parenteral routes. The term “parenteral” includes subcutaneous, intravenous, intramuscular, or infusion. Direct injection could also be preferred for local delivery to the site of injury. Oral administration may be preferred for prophylactic treatment because of the convenience to the patient as well as the dosing schedule.

The compositions may conveniently be presented in unit dosage form and may be prepared by any of the methods well known in the art of pharmacy. All methods include the step of bringing the 12-HETrE analog into association with a carrier which constitutes one or more accessory ingredients. In general, the compositions are prepared by uniformly and intimately bringing the 12-HETrE analog into association with a liquid carrier, a finely divided solid carrier, or both, and then, if necessary, shaping the product.

Compositions suitable for parenteral administration conveniently comprise a sterile aqueous preparation of the 12-HETrE analog, which is preferably isotonic with the blood of the recipient. This aqueous preparation may be formulated according to known methods using those suitable dispersing or wetting agents and suspending agents. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono or di-glycerides. In addition, fatty acids such as oleic acid find use in the preparation of injectables. Carrier formulations suitable for oral, subcutaneous, intravenous, intramuscular, etc. can be found in Remington's Pharmaceutical Sciences, Mack Publishing Company, Easton, Pa.

Compositions suitable for oral administration may be presented as discrete units such as capsules, cachets, tablets, or lozenges, each containing a predetermined amount of the 12-HETrE analog. Other compositions include suspensions in aqueous liquors or non-aqueous liquids such as a syrup, an elixir, or an emulsion.

The 12-HETrE agonist and angiogenic factor or other therapeutic useful in the treatment of inflammatory or cardiovascular disorders including ischemic diseases may be administered by the same method, e.g. intravenous, oral, etc. or may be administered separately by different modes, e.g., 12-HETrE agonist administered orally, angiogenic factor administered intravenously, etc. In one embodiment of the invention, the 12-HETrE agonist and the angiogenic factor or other therapeutic are co-administered intravenously. In another embodiment the 12-HETrE agonist and the angiogenic factor or other therapeutic are administered separately.

Other delivery systems can include time-release, delayed release or sustained release delivery systems. Such systems can avoid repeated administrations of the 12-HETrE analog of the invention, increasing convenience to the subject and the physician. Many types of release delivery systems are available and known to those of ordinary skill in the art. They include polymer based systems such as polylactic and polyglycolic acid, polyanhydrides and polycaprolactone; nonpolymer systems that are lipids including sterols such as cholesterol, cholesterol esters and fatty acids or neutral fats such as mono-, di- and tri-glycerides; liposomes; phospholipids; hydrogel release systems; silastic systems; peptide based systems; wax coatings, compressed tablets using conventional binders and excipients, partially fused implants and the like. Specific examples include, but are not limited to: (a) erosional systems in which the polysaccharide is contained in a form within a matrix, found in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

Use of a long-term sustained release implant may be particularly suitable for treatment of chronic conditions. “Long-term” release, as used herein, means that the implant is constructed and arranged to deliver therapeutic levels of the active ingredient for at least 7 days, and preferably 30-60 days. The implant may be positioned at the site of injury. Long-term sustained release implants are well known to those of ordinary skill in the art and include some of the release systems described above.

Each of the foregoing patents, patent applications and references that are recited in this application are herein incorporated in their entirety by reference. Having described the presently preferred embodiments, and in accordance with the present invention, it is believed that other modifications, variations and changes will be suggested to those skilled in the art in view of the teachings set forth herein. It is, therefore, to be understood that all such variations, modifications, and changes are believed to fall within the scope of the present invention as defined by the appended claims.

### EXAMPLES

### Example 1

### Synthesis of N-(Methylsulfonyl)-12(R)-hydroxyeicosa-5(Z),8(Z),14(Z)-trienamide (1-a-(R))

(1) n-BuLi (2.1M in hexane, 13.5 mL, 28.35 mmol) was slowly added to a stirring, −78° C. solution of benzyl alcohol (3.062 g, 28.35 mmol) in dry tetrahydrofuran (THF) (50 mL) under an argon atmosphere. After 30 minutes, a solution of (S)-(+)-dihydro-5-(p-tolylsulfonyloxymethyl)-2(3H)-furanone (5.67 g, 21.81 mmol) in THF (60 mL) was added at −78° C. to the reaction mixture and the stirring was continued at 0° C. for 1 h. The reaction was quenched by the addition of saturated aqueous NH₄Cl solution (20 mL) followed by brine (20 mL). The reaction mixture was extracted with AcOEt (30 mL×3). The combined organic extracts were dried over anhydrous MgSO₄, then solvent and volatiles were removed under vacuum, and the residue was purified by column chromatography using AcOEt/hexane (3:50) to give 1-1 as a pale yellow oil (3.60 g, 80%).

¹H NMR (CDCl₃, 300 MHz) δ ppm: 1.76-1.85 (m, 1H), 1.93-2.05 (m, 1H), 2.47-2.54 (m, 3H), 2.74 (t, J=4.6 Hz, 1H), 2.94-3.01 (m, 1 H), 5.07 (s, 2H), 7.33-7.41 (m, 5 H).

(2) n-BuLi (2.38 mL, 5.0 mmol) was added to a stirring solution of 1(Z)-iodo-1-heptene [U. Ravid, R. M. Silverstein, L. R. Smith Tetrahedron 34, 1978, 1449] (1.125 g, 5.0 mmol) in ether (10 mL) at −78° C. under argon. After 30 minutes, the resulting vinyl lithium reagent was added via cannula to a stirring, −78° C. suspension of CuCN (0.203 g, 2.40 mmol) in ether (10 mL). The temperature of the cooling bath was slowly raised to −30° C. during which time the reaction solution become pale green and homogeneous. The reaction mixture was re-cooled to −78° C. and 1-1 (0.412 g, 2.0 mmol) in ether (10 mL) was added to the cuprate solution during which time the reaction mixture developed a bright yellow coloration. After another 30 minutes, the reaction mixture was quenched by addition of methanol (5 mL), then slowly brought to 0° C. whereupon aqueous NH₄OH solution (5 mL) and 10% aqueous NH₄OH solution (2mL) were added. The mixture was stirred at room temperature for 30 minutes and then extracted with ether (10 mL×3). The combined organic extracts were dried over anhydrous MgSO₄, then solvent and volatiles were removed under vacuum, and the residue was purified by column chromatography using AcOEt/hexane (1:20) to yield 1-2 (0.350 g, 89%).

¹H NMR (CDCl₃, 300 MHz) δ ppm: 0.86 (t, J=6.9 Hz, 3H), 1.22-1.36 (m, 6H), 1.81-1.94 (m, 1H), 1.98-2.04 (m, 2H), 2.21-2.56 (m, 5H), 4.45-4.54 (m, 1H), 5.28-5.37 (m, 1H), 5.50-5.59 (m, 1H).

(3) Diisobutylaluminum hydride (DiBAL) in toluene (1.84 mL of a 1 M solution, 1.84 mmol) was added dropwise over 5 minutes to a stirring, −78° C. solution of 1-2 (0.300 g, 1.53 mmol) in dry toluene (10 mL) under an argon atmosphere. After 1.5 h, the reaction mixture was cautiously treated with methanol (2 mL), then warmed to room temperature and diluted with a 1:1 mixture of AcOEt/hexane (20 mL). A saturated, aqueous solution of citric acid (10 mL) was added and the reaction mixture was extracted with AcOEt/hexane (1:1, 10 mL×3). The combined organic extracts were washed with brine, dried over anhydrous MgSO₄, and all volatiles were removed under vacuum. Column chromatography of the residue over a silica gel using AcOEt/hexane (1:4) as eluant afforded 1-3 (0.276 g, 91%) as a mixture of anomers.

¹H NMR (CDCl₃, 400 MHz) δ ppm: 0.89 (t, J=6.8 Hz, 3H), 1.26-1.37 (m, 6H), 1.46-2.52 (m, 8H), 2.85 (d, J=3.0 Hz, 0.41H), 2.90 (d, J=3.0 Hz, 0.59H), 4.00-4.08 (m, 0.41H), 4.22-4.28 (m, 0.59H), 5.34-5.57 (m, 3H).

(4) (Me₃SI)₂Nli (2.12 mL, 1.0 M in hexane, 2.12 mmol) was added to a 0° C. solution of (Z)-(7-methoxycarbonyl-hept-3-enyl)triphenylphosphonium bromide [A. V. Rama Rao, A. V. Purandare, A. K. Singh, Bioorganic & Medicinal Chemistry Letters, 1991, 1, 201] (1.0879 g, 2.120 mmol) in THF (4.9 mL) and hexamethylphosphoramide (HMPA, 3.0 mL). After 45 min, the reaction mixture was cooled to −78° C. and stirred for 4.5 h before a solution of 1-3 (220 mg, 1.0 mmol, azeotropically dried with benzene) in THF (2.0 mL) was added. The reaction mixture was gradually warmed up to room temperature. After stirring overnight, the reaction mixture was poured into a saturated, aqueous solution of NH₄Cl (50 mL), extracted with hexane/AcOEt (3:1, 50 mL×2), washed with H₂O (40 mL), brine (40 mL), dried (MgSO₄), and concentrated under vacuum. The crude residue was purified by silica gel column chromatography (gradient from 5% to 9% EtOAc/hexane) to give 1-4-(S) (98.4 mg, 29% [73% based on recovered starting material]) as a pale yellow oil accompanied by recovered 1-3 (119 mg, 0.6 mmol, 60%).

¹H NMR (CDCl₃, 400 MHz) δ ppm: 0.89 (t, J=6.8 Hz, 3H), 1.18-1.42 (m, 6H), 1.65-1.79 (m, 2H), 2.00-2.29 (m, 8H), 2.33 (t, J=7.6 Hz, 2H), 2.74-2.86 (m, 2H), 3.59-3.71 (m, 1H), 3.67 (s, 3H) 5.31-5.46 (m, 5H), 5.52-5.62 (m, 1H); ¹³C NMR (CDCl₃, 100 MHz) δ ppm: 14.4, 23.0, 24.1, 25.2, 26.0, 26.9, 27.8, 29.7, 31.9, 33.8, 35.8, 37.0, 51.9, 71.4, 125.5, 128.7, 129.2, 129.5, 130.1, 133.8, 174.5.

(5) Benzoic acid (32.5 mg, 0.266 mmol), Ph₃P (69.9 mg, 0.266 mmol), and diethyl azodicarboxylate (DEAD) (41.9 μL, 0.266 mmol) were added sequentially to a 0° C. solution of 1-4-(S) (49.8 mg, 0.148 mmol) in THF (7.4 mL). After stirring for 1.5 h, all volatiles were removed under vacuum and the residue was dissolved in AcOEt (30 mL), washed with brine (30 mL×2), dried (MgSO₄), and concentrated under vacuum. The crude residue was purified by silica gel column chromatography (gradient from 1% to 5% EtOAc/hexane) to give 1-5 (56.8 mg, 87%) as a pale yellow oil.

¹H NMR (CDCl₃, 400 MHz) δ ppm: 0.86 (t, J=6.6 Hz, 3H), 1.16-1.38 (m, 6H), 1.92-2.09 (m, 4H), 2.10-2.22 (m, 2H), 2.26 (t, J=7.8 Hz, 2H), 2.36-2.54 (m, 2H), 2.64-2.78 (m, 2H), 3.65 (s, 3H), 5.10-5.29 (m, 1H), 5.26-5.54 (m, 6H), 7.40-7.48 (m, 2H), 7.50-7.59 (m, 1H), 8.01-8.08 (m, 2H)

(6) NaOMe (0.59 mL, ˜25% in MeOH, ˜2.58 mmol) was added to a solution of 1-5 (56.8 mg, 0.129 mmol) in MeOH (6.4 mL). After stirring overnight, the reaction mixture was acidified to pH=5.5 using 1.0 M aqueous oxalic acid (2.0 mL), all volatiles were removed under vacuum and the residue was diluted with brine (30 mL), extracted with AcOEt (30 mL×2). The combined organic extracts were washed with H₂O (30 mL×2), brine (30 mL×2), dried (MgSO₄) and concentrated. The crude residue was dissolved in MeOH (10 mL) and a solution of CH₂N₂ in Et₂O was added at 0° C. until the yellow coloration of the diazomethane persisted for several min. After stirring for 30 min at 0° C., the reaction mixture was concentrated under vacuum and the residue was purified by silica gel column chromatography (gradient from 6% to 8% EtOAc/hexane) to give 1-4-(R) (39.0 mg, 90%) as a pale yellow oil.

¹H NMR (CDCl₃, 400 MHz) δ ppm: 0.89 (t, J=6.8 Hz, 3H), 1.22-1.41 (m, 6H), 1.47-1.61 (m, 2H), 1.65-1.79 (m, 3H), 2.00-2.29 (m, 8H), 2.33 (t, J=7.6 Hz, 2H), 2.80 (t, J=6.2 Hz, 2H), 3.59-3.71 (m, 1H), 3.67 (s, 3H), 5.31-5.45 (m, 5H), 5.52-5.61 (m, 1H); ¹³C NMR (CDCl₃, 100 MHz) δ ppm: 14.3, 22.8, 23.9, 25.0, 25.8, 26.7, 27.6, 29.5, 31.7, 33.6, 35.6, 36.7, 51.7, 71.2, 125.2, 128.5, 129.0, 129.2, 129.8, 133.7, 174.4.

(7) A 1 M aqueous solution of LiOH (0.35 mL, 0.35 mmol) was added to a solution of 1-4-(R) (39.0 mg, 0.116 mmol) in THF (4.6 mL) and H₂O (0.81 mL). After stirring overnight, then reaction mixture was acidified to pH 4.5 with 1 M aqueous oxalic acid, the THF was evaporated under vacuum, and the remaining aqueous phase was diluted with H₂O (30 mL), extracted with AcOEt (30 mL×2). The combined organic extracts were washed with brine (30 mL×2), dried (MgSO₄), concentrated under vacuum, and the residue was purified by silica gel column chromatography to give 1-6-(R) (37.3 mg, 100%) as a pale yellow oil.

¹H NMR (CDCl₃, 400 MHz) δ ppm: 0.89 (t, J=6.8 Hz, 3H), 1.22-1.41 (m, 6H), 1.46-1.62 (m, 2H), 1.65-1.75 (m, 2H), 1.98-2.32 (m, 8H), 2.36 (t, J=7.2 Hz, 2H), 2.73-2.88 (m, 2H), 3.62-3.72 (m, 1H), 5.31-5.47 (m, 5H), 5.51-5.61 (m, 1H), 6.65 (br s, 1H); ¹³C NMR (CDCl₃, 100 MHz) δ ppm: 14.2, 22.7, 23.8, 24.8, 25.8, 26.6, 27.6, 29.5, 31.7, 33.4, 35.5, 36.6, 71.6, 125.0, 128.5, 128.8, 129.4, 129.6, 133.7, 178.9.

(8) 1-[3-(Dimethylamino)propyl]-3-ethylcarbodiimide hydrochloride (EDC.HCl) (32.4 mg, 0.169 mmol) was added to a 0° C. solution of 1-6-(R) (21.8 mg, 0.068 mmol) and N-hydroxysuccinimide (19.5 mg, 0.169 mmol) in THF (5 mL). After stirring overnight, another portion of EDC.HCl (3 8.1 mg, 0.199 mmol) was added and the reaction mixture was allowed to stir overnight, then quenched with water (30 mL), and extracted with AcOEt (30 mL×2). The combined organic extracts were washed with H₂O (30 mL×2), brine (30 mL×2), dried (MgSO₄), and concentrated under vacuum. The resultant crude NIH ester was dissolved in THF (63.4 mL) and methanesulfonamide (12.9 mg, 0.136 mmol) and 1,8-diazabicyclo[5.4.0]undec-7-ene (DBU) (20.3 μL, 0.136 mmol) were added. After stirring at room temperature for 3 days, 1 N aqueous HCl (30 mL) was added and the reaction mixture was extracted with AcOEt (30 mL×2). The combined organic extracts were washed with brine (30 mL×2), dried (MgSO₄), and concentrated under vacuum. The crude residue was purified by silica gel column chromatography (gradient from 10% to 80% EtOAc/hexane) to give N-(methylsulfonyl)-12(R)-hydroxyeicosa-5(Z),8(Z),14(Z)-trienamide (1-a-(R)) (22.8 mg, 84% over 2 steps) as a pale yellow oil.

¹H NMR (CDCl₃, 400 MHz) δ ppm: 0.89 (t, J=6.8 Hz, 3H), 1.22-1.41 (m, 6H), 1.46-1.82 (m, 5H), 1.96-2.40 (m, 8H), 2.32 (t, J=7.6 Hz, 2H), 2.80 (t, J=6.8 Hz, 2H), 3.27 (s, 3H), 3.64-3.74 (m, 2H), 5.27-5.49 (m, 5H), 5.53-5.64 (m, 1H), 9.31 (br s, 1H); ¹³C NMR (100 MHz, CDCl₃) δ ppm: 14.3, 22.7, 23.9, 24.1, 25.9, 26.6, 27.6, 29.5, 31.7, 35.6, 35.9, 36.5, 41.6, 71.8, 124.8, 128.4, 128.5, 129.7, 129.8, 134.0, 172.6.

### Example 2

### Synthesis of N-(Methylsulfonyl)-12(S)-hydroxyeicosa-5(Z),8(Z),14(Z)-trienamide (1-a-(S))

(1) The reaction was carried out substantially in the same manner as in Example 1(7), but using 1-4-(S), obtained in Example 1 (4), to afford 12(S)-(5Z,8Z,14Z)-12-hydroxyeicosa-5,8,14-trienoic acid (1-6-(S)).

Spectral data (¹H NMR and ¹³C NMR) were identical with those of 1-6-(R).

### Example 3

### Synthesis of 12(R)-Methyoxyeicosa-5(Z),8(Z),14(Z)-trienoic acid (2-a-(R))

(1) BF₃.Et₂O (50 μL, 0.395 mmol) was added to a −78° C. solution of the compound 1-4-(R) (32.1 mg, 0.0954 mmol), obtained in Example 1(6), in CH₂Cl₂ (5.0 mL). After stirring for 10 min at −78° C., a saturated ethereal solution of CH₂N₂ (ca. 5 mL) was added until the yellow coloration of excess diazomethane persisted for several min. The reaction mixture was stirred for 30 min and additional CH₂N₂ in Et₂O (ca. 2 mL) was added at −78° C. After stirring for another 30 min at −78° C., a saturated aqueous solution of NaHCO₃ (30 mL) was added and the reaction mixture was warmed to room temperature, filtered to remove insoluble products, then extracted with AcOEt (50 mL×2). The combined organic extracts were washed with a saturated aqueous solution of NaHCO₃ (30 mL), dried (MgSO₄), filtered, and concentrated under vacuum. The crude residue was purified by silica gel column chromatography (gradient from EtOAc to 2% EtOAc/hexane) to give 2-1-(R) (30.5 mg, 91%) as a colorless oil.

¹H NMR (CDCl₃, 400 MHz) δ ppm: 0.89 (t, J=6.9 Hz, 3H), 1.20-1.41 (m, 6H), 1.45-1.60 (m, 2H), 1.62-1.76 (m, 2H), 1.98-2.36 (m, 8H), 2.32 (t, J=7.6 Hz, 2H), 2.69-2.84 (m, 2H), 3.14-3.24 (m, 1H), 3.35 (s, 3H), 3.67 (s, 3H), 5.28-5.52 (m, 6H); ¹³C NMR (CDCl₃, 100 MHz) δ ppm: 14.3, 22.8, 23.4, 25.0, 25.8, 26.7, 27.6, 29.5, 31.2, 31.8, 33.6, 33.7, 51.7, 56.8, 80.5, 125.2, 128.3, 129.0, 129.3, 130.0, 132.3, 174.3; MS (CI, CH₄): 351 (M+H)⁺.

(2) Using the compound obtained in the above (1), the reaction was carried out in the same manner as in Example 1(7) to afford title compound, 2-(R).

¹H NMR (CDCl₃, 400 MHz) δ ppm: 0.89 (t, J=6.9 Hz, 3H), 1.20-1.42 (m, 6H), 1.47-1.58 (m, 2H), 1.64-1.78 (m, 2H), 1.96-2.43 (m, 8H), 2.36 (t, J=7.3 Hz, 2H), 2.69-2.90 (m, 2H), 316-3.28 (m, 1H), 3.38 (s, 3H), 5.28-5.53 (m, 6H); ¹³C NMR (CDCl₃, 100 MHz) δ ppm: 14.2, 22.8, 23.5, 24.8, 25.8, 26.6, 27.6, 29.5, 31.1, 31.8, 33.4, 33.7, 56.7, 80.9, 125.1, 128.3, 128.8, 129.5, 129.8, 132.4, 179.0; MS (CI, CH₄): 337 (M+H)⁺.

### Example 4

### Synthesis of 12(S)-Methoxyeicosa-5(Z),8(Z),14(Z)-trienoic acid (2-a-(S))

(1) The reaction was carried out substantially in the same manner as in Example 3 (1), but using 1-4-(S), obtained in Example 1 (4), to afford methyl 12(S)-methoxyeicosa-5(Z),8(Z),14(Z)-trienoate, 2-1-(S).

The spectral data (¹H NMR, ¹³C NMR, and MS) were identical with those of 2-1-(R).

(2) Using the compound obtained in the above (1), the reaction was carried out in the same manner as in Example 1 (7) to afford title compound, 2-(S).

The spectral data of 2-(S) (¹H NMR, ¹³C NMR, and MS) were identical with those of 2-(R).

### Example 5

### Synthesis of 12(R)-hydroxyeicosa-8(Z),14(Z)-dien-5-ynoic acid (3-(R))

(1) Ph₃P (6.513 g, 24.8 mmol) was added portionwise to a 0° C. mixture of methyl 8-hydroxy-oct-5-ynoate [Julian Adams and Joshua Rokach, Tetrahedron Lett. 1984, 25, 35.] (4.025 g, 23.6 mmol) and CBr₄ (8.235 g, 24.8 mmol) in CH₂Cl₂ (47 mL). After stirring for 2 h, the mixture was concentrated in vacuo and the residue was purified by silica gel column chromatography (gradient from 2% to 8% EtOAc/hexane) to give 3-1 (5.226 g, 95%) as a pale yellow oil.

¹H NMR (CDCl₃, 400 MHz) δ ppm: 1.77-1.87 (m, 2H), 2.19-2.28 (m, 2H), 2.45 (t, J=7.6 Hz, 2H), 2.67-2.76 (m, 2H), 3.41 (t, J=7.2 Hz, 2H), 3.68 (s, 3H).

(2) A mixture of 3-1 (2.481 g, 10.6 mmol) and Ph₃P (2.931 g, 11.2 mmol) in dry CH₃CN (10.6 mL) was heated in a sealed tube at 90° C. under an argon atmosphere for 90 h. Upon cooling, the solvent was evaporated under vacuum and the residue was dissolved in dry CH₂Cl₂ (20 mL), then diluted with dry Et₂O (80 mL) and decanted. The residual white solid was washed with dry Et₂O (80 mL×2) and dried under reduced pressure to give 3-2 (5.108 g, 97%) as a white powder.

¹H NMR (CDCl₃, 400 MHz) ppm: 1.44-1.56 (m, 2H), 1.67-86 (m, 2H), 2.22 (t, J=7.2 Hz, 2H), 2.76-2.92 (m, 2H), 3.67 (s, 3H), 4.06-4.20 (m, 2H), 7.65-7.94 (m, 15H).

(3) The reaction was carried out substantially in the manner as in Example 1 (4), but using the compound 3-2 obtained in the above (2) to afford 3-3-(S).

¹H NMR (CDCl₃, 400 MHz) δ ppm: 0.89 (t, J=6.8 Hz, 3H), 1.22-1.41 (m, 6H), 1.49-1.60 (m, 2H), 1.67 (d, J=4.0 Hz, 1H), 1.76-1.85 (m, 2H), 1.98-2.11 (m, 2H), 2.14-2.29 (m, 6H), 2.43 (t, J=7.4 Hz, 2H), 2.85-2.99 (m, 2H), 3.58-3.70 (m, 1H), 3.68 (s, 3H), 5.34-5.50 (m, 3H), 5.52-5.62 (m, (1H); ¹³C NMR (CDCl₃, 100 MHz) δ ppm: 4.2, 17.3, 18.4, 22.7, 23.7, 24.3, 27.6, 29.5, 31.7, 33.1, 35.6, 36.4, 51.7, 70.9, 78.9, 79.6, 125.1, 125.5, 131.0, 133.7, 173.9; [α]_{D}²⁶+2.87° (c 2.01, MeOH)

(4) The reaction was carried out substantially in the similar manner as in Example 1 (5), but using the compound 3-3-(S) obtained in the above (3) to afford 3-4.

¹H NMR (CDCl₃, 400 MHz) δ ppm: 0.86 (t,J=6.8 Hz, 3H), 1.17-1.36 (m, 6H), 1.68-1.86 (m, 4H), 1.92-2.27 (m, 6H), 2.36-2.54 (m, 2H), 2.41 (t, J=7.6 Hz, 2H), 2.81-2.90 (m, 2H), 3.66 (s, 3H), 5.09-5.19 (m, 1H), 5.35-5.54 (m, 4H), 7.40-7.48 (m, 2H), 7.51-7.59 (m, 1H), 8.01-8.07 (m, 2H); ¹³C NMR (CDCl₃, 400 MHz) δ ppm: 14.2, 17.3, 18.4, 22.7, 23.4, 24.3, 27.5, 29.4, 31.7, 32.2, 33.0, 33.5, 51.7, 74.2, 78.9, 79.3, 123.9, 125.8, 128.5(2C), 129.7(2C), 130.2, 130.8, 132.9, 133.3, 166.3, 173.9

(5) The reaction was carried out substantially in the manner as in Example 1 (6), but using the compound 3-4 obtained in the above (4) to afford 3-3-(R).

The spectral data of 3-3-(R) (¹H NMR, ¹³C NMR) were identical with those of 3-3-(S). [α]_{D}²⁶−3.09° (c 2.04, MeOH)

(6) The reaction was carried out substantially in the similar manner as in Example 1 (7), but using the compound 3-3-(R), obtained in the above (4), to afford the compound 3-(R).

¹H NMR (CDCl₃, 400 MHz) δ ppm: 0.89 (t, J=6.6 Hz, 3H), 1.21-1.41 (m, 6H), 1.49-1.61 (m, 2H), 1.76-1.86 (m, 3H), 1.98-2.30 (m, 8H), 2.47 (t, J=7.2 Hz, 2H), 2.85-2.99 (m, 2H), 3.59-3.70 (m, 1H), 5.34-5.49 (m, 3H), 5.50-5.61 (m, 1H), 6.34 (br s, 2H); ¹³C NMR (CDCl₃, 400 MHz) δ ppm: 14.2, 17.3, 18.4, 22.7, 23.6, 24.0, 27.6, 29.5, 31.7, 33.0, 35.5, 36.3, 71.1, 78.6, 79.8, 125.0, 125.5, 130.9, 133.8, 179.2.

### Example 6

### Synthesis of 12(S)-Hydroxyeicosa-8(Z),14(Z)-dien-5-ynoic acid (3-(S))

The reaction was carried out substantially in the same manner as in Example 1 (7), but using the compound 3-3-(S) obtained in Example 5 (3) to afford title compound, 3-(S).

The spectral data (¹H NMR, ¹³C NMR) were identical with those of 3-(R).

### Example 7

### Synthesis of (9(R)-Hydroxyheptadeca-2(Z),5(Z),11(Z)-trienyloxy)acetic acid (4-a(R))

(1) n-Bu₄NHSO₄ (45.0 mg, 0.13 mmol) was added to a room temperature solution of 5-(tetrahydropyran-2-yloxy)pent-2-yn-1-ol [Michel Cantin, Rolf Schütz and Christian J. Leumann Tetrahedron Lett. 1997, 38 4211] 2.444 g, 13.3 mmol) and t-butyl bromoacetate (3.105 g, 15.9 mmol) in a mixture of toluene (30 mL) and aqueous NaOH (30 mL, 1.0 M). After stirring vigorously for 1.5 h at room temperature, the organic layer was separated and the aqueous layer was extracted with Et₂O (100 mL). The combined organic layers were dried (MgSO₄), filtered and concentrated under vacuum. The crude residue was purified by silica gel column chromatography (gradient from 5% to 20% EtOAc/hexane) to give compound 4-1 (3.657 g, 92%) as a pale yellow oil.

¹H NMR (CDCl₃, 400 MHz) δ ppm: 1.40-1.90 (m, 6H), 1.48 (s, 9H), 2.54 (tt, J=7.1, 2.1 Hz, 2H), 3.47-3.60 (m, 2H), 3.78-3.92 (m, 2H), 4.06 (s, 2H), 4.27 (t, J=2.1 Hz, 2H), 4.62-4.66 (m, 1H).

(2) NaBH₄ (23.2 mg, 0.61 mmol) was added to a room temperature solution of Ni(OAc)₂∘4H₂O (152 mg, 0.61 mmol) in EtOH (40 mL) under a H₂ blanket (1 atmosphere). After stirring for 30 min at room temperature, ethylenediamine (81.9 μL, 1.23 numol) was added followed by addition of the compound obtained in the above (1), 4-1 (3.657 g, 12.3 mmol) in EtOH (20 mL). After stirring for 2 h, reaction mixture was diluted with Et₂O (60 mL) and filtered through a bed of silica gel. Solvent was removed in vacuo to give the compound 4-2 (3.532 g, 96%) as a colorless oil, which was used to the next reaction without further purification.

¹H NMR (CDCl₃, 400 MHz) δ ppm: 1.40-1.89 (m, 6H), 1.48 (s, 9H), 2.32-2.45 (m, 2H), 3.36-3.55 (m, 2H), 3.70-3.90 (m, 2H), 3.96 (s, 2H), 4.17 (d, J=5.2 Hz, 2H), 4.56-4.61 (m, 1H), 5.61-5.73 (m, 2H)

(3) Pyridiniump-toluenesulfonate (PPTS) (88.7 mg, 0.35 mmol) was added to a solution of the compound obtained in the above (2), 4-2 (3.532 g, 11.8 mmol) in MeOH (39 mL). After stirring for 4 days, the reaction mixture was concentrated in vacuo and the residue was purified by silica gel column chromatography (gradient from 10 to 60% EtOAc/hexane) to give the compound 4-3 (2.423 g, 95%) as a pale yellow oil.

¹H NMR (CDCl₃, 400 MHz) δ ppm: 1.49 (s, 9H), 1.75 (t, J=5.6 Hz, 1H), 2.33-2.46 (m, 2H), 3.64-3.71 (m, 2H), 3.97 (s, 2H), 4.15 (d, J=6.4 Hz, 2H), 5.64-5.83 (m, 2H).

(4) The reaction was carried out substantially in the manner as in Example 5 (1), but using the compound 3-3 obtained in the above (3) to afford 4-4.

¹H NMR (CDCl₃, 400 MHz) δ ppm: 1.49 (s, 9H), 2.61-2.66 (m, 2H), 3.40 (t, J=6.9 Hz, 2H), 3.97(s, 2H), 4.16 (d, J=6.4 Hz, 2H), 5.58-5.79 (m, 2H); ¹³C NMR (CDCl₃, 100 MHz) δ ppm: 27.7 (3C), 30.5, 31.8, 66.0, 67.2, 80.8, 128.0, 129.9, 169.0.

(5) The reaction was carried out substantially in the manner as in Example 5 (2), but using the compound 4-4 obtained in the above (4) to afford 4-5.

¹H NMR (CDCl₃, 400 MHz) δ ppm: 1.42 (s, 9H), 2.52-2.65 (m, 2H), 3.86 (s, 2H), 3.92-4.04 (m, 4H), 5.57-5.68 (m, 1H), 5.85-5.97 (m, 1H), 7.64-7.95 (m, 15H).

(6) The reaction was carried out substantially in the manner as in Example 1 (4), but using the compound 4-5 obtained in the above (5) to afford 4-6-(S).

¹H NMR (CDCl₃, 400 MHz) δ ppm: (t, J=6.9 Hz, 3H), 1.22-1.42 (m, 6H), 1.45-1.60 (m, 2H), 1.48 (s, 9H), 1.73 (d, J=4.3 Hz, 1H), 2.00-2.10 (m, 2H), 2.12-2.29 (m, 4H), 2.80-2.93 (m, 2H), 3.57-3.67 (m, 1H), 3.96 (s, 2H), 4.12-4.22 (m, 2H), 5.31-5.48 (m, 3H), 5.52-5.67 (m, 3H); ¹³C NMR (CDCl₃, 100 MHz) δ ppm: 14.1, 22.6, 23.7, 26.0, 27.5, 28.2, (3C), 29.4, 31.6, 35.6, 36.5, 66.6, 67.7, 70.9, 81.6, 125.2, 125.4, 127.6, 130.4, 132.7, 133.4, 169.8; MS (CI, CH₄): 381 (M+H)⁺.

(7) The reaction was carried out substantially in the manner as in Example 1 (5), but using the compound 4-6-(S) obtained in the above (6) to afford 4-7.

¹H NMR (CDCl₃, 400 MHz) δ ppm: 0.86 (t, J=6.9 Hz, 3H), 1.18-1.86 (m, 8H), 1.47 (s, 9H), 1.98-2.08 (m, 2H), 2.10-2.22 (m, 2H), 2.36-2.54 (m, 2H), 2.71-2.87 (m, 2H), 3.90 (s, 2H), 4.10 (d, J=4.9 Hz, 2H), 5.10-5.20 (m, 1H), 5.27-5.62 (m, 6H), 7.40-7.48 (m, 2H), 7.51-7.59 (m, 1H), 7.99-8.11 (m, 2H); ¹³C NMR (CDCl₃, 100 MHz) δ ppm: 14.2, 22.7, 23.5, 26.0, 27.5, 28.3, (3C), 29.4, 31.7, 32.2, 33.7, 66.7, 67.8, 74.2, 81.7, 124.0, 125.7, 128.0, 128.5 (2C), 129.7 (3C), 130.8, 132.5; 133.0, 133.3, 166.3, 169.8.

(8) The reaction was carried out substantially in the manner as in Example 1 (7), but using the compound 4-7 obtained in the above (7) to afford the title compound 4-a-(R) (32%) and 4-8 (41%).

### 9(R)-Hydroxyheptadeca-2(Z),5(Z),11(Z)-trienyloxy)acetic acid (4-a-(R))

¹H NMR (CDCl₃, 400 MHz) δ ppm: 0.88 (t, J=6.9 Hz, 3H), 1.15-1.65 (m, 8H), 1.94-2.35 (m, 6H), 2.78-2.93 (m, 2H), 3.57-3.70 (m, 1H), 4.03 (s, 2H), 4.10-4.26 (m, 2H), 5.22-5.92 (m, 6H); MS(CI, CH₄): 325(M+H)⁺.

### 9(R)-Benzoyloxyheptadeca-2(Z),5(Z),11(Z)-trienyloxy)acetic acid (4-8)

¹H NMR (CDCl₃, 400 MHz) δ ppm: 0.85 (t, J=6.9 Hz, 3H), 1.14-1.40 (m, 6H), 1.65-2.85 (m, 2H), 1.96-2.20 (m, 4H), 2.36-2.54 (m, 2H), 2.68-2.83 (m, 2H), 3.99 (s, 2H), 4.09 (d, J=6.2 Hz, 2H), 5.10-5.21 (m, 1H), 5.26-5.71 (m, 6H), 7.38-7.59 (m, 3H), 7.99-8.09 (m, 2H).

### Example 8

### Synthesis of 9(S)-Hydroxyheptadeca-2(Z),5(Z),11(Z)-trienyloxy)acetic acid (4-a-(S))

The reaction was carried out substantially in the manner as in Example 1 (7), but using the compound 4-6-(S) obtained in Example 7 (6) to afford the title compound 4-a-(S). The spectral data (¹H NMR, MS) were identical with those of 4-a-(R).

### Example 9

### Synthesis of 12(R)-Hydroxyeicosa-8(Z),14(Z)-dienoic acid (5-(R))

(1) The reaction was carried out substantially in the manner as in Example 5 (2), but using methyl 8-bromooctanoate, which had been prepared by conventional esterification of 8-bromooctanoic acid, to afford 5-1.

¹H NMR (CDCl₃, 300 MHz) δ ppm: 1.19-1.38 (m, 4H), 1.45-1.76 (m, 6H), 2.24 (t, J=7.6 Hz, 2H), 3.62 (s, 3H), 3.63-3.78 (m, 2H), 7.65-7.96 (m, 15H).

(2) The reaction was carried out substantially in the manner as in Example 1 (4), but using the compound 5-1 obtained in the above (1), to afford 5-2-(S).

¹H NMR (CDCl₃, 400 MHz) δ ppm: 0.89 (t, J=6.8 Hz, 3H), 1.26-1.37 (m, 12H), 1.50-1.56 (m, 2H), 1.61-1.65 (m, 2H), 2.01-2.25 (m, 6H), 2.23 (t, J=6.8 Hz, 2H), 2.31 (t, J=8.0 Hz, 2 H), 3.60-3.66 (m, 1H), 3.67 (s, 3H), 5.37-5.43 (m, 3H), 5.54-5.61 (m, 1H); ¹³C NMR (CDCl₃, 75.3 MHz) δ ppm: 8.53, 17.03, 18.13, 19.36, 21.57, 21.87, 23.32, 23.47, 23.82, 23.93, 25.99, 28.54, 29.86, 31.15, 45.93, 65.60, 119.49, 123.77, 124.85, 127.97, 168.79.

(3) The reaction was carried out substantially in the manner as in Example 1 (5), but using the compound 5-2-(S), obtained in the above (2), to afford 5-3.

¹H NMR (CDCl₃, 400 MHz) δ ppm: 0.86 (t, J=6.7 Hz, 3H), 1.20-1.36 (m, 12H), 1.54-1.62 (m, 2H), 1.69-1.81 (m, 2H), 1.94-1.99 (m, 2H), 2.00-2.06 (m, 2H), 2.10-2.15 (m, 2H), 2.27 (t, J=7.6 Hz, 2H), 2.38-2.50 (m, 2H), 3.66 (s, 3H), 5.15-5.18 (m, 1H), 5.34-5.52 (m, 4H), 7.44 (t, J −7.6 HZ, 2H), 7.55 (t, J=7.6 Hz, 1H), 8.04 (d, J=7.6 Hz, 2H).

(4) The reaction was carried out substantially in the manner as in Example 1 (6), but using the compound 5-3, obtained in the above (3), to afford 5-2-(R).

¹H NMR of 5-2-(R) was identical with that of 5-2-(S).

(5) The reaction was carried out substantially in the manner as in Example 1 (7), but using the compound 5-2-(R), obtained in the above (4), to afford the title compound 5-(R).

¹H NMR (CDCl₃, 400 MHz) δ ppm: 0.89 (t, J=6.8 Hz, 3H), 1.27-1.35 (m, 12H), 1.48-1.57 (m, 2H), 1.59-1.68(m, 2H), 2.00-2.26 (m, 8H), 2.34 (t, J=7.2 Hz, 2H), 3.66 (quint, J=5.6 Hz, 1H), 5.33-5.43 (m, 3H), 5.54-5.60 (m, 1H).

### Example 10

### Synthesis of 12(S)-Hydroxyeicosa-8(Z),14(Z)-dienoic acid (5-(S))

The reaction was carried out substantially in the manner as in Example 1 (7), but using the compound 5-2-(S), obtained in Example 9 (2), to afford the title compound 5-(S).

¹H NMR of 5-(S) was identical with that of 5-(R).

### Example 11

### Synthesis of 12(S)-Hydroxyeicosa-5(Z),8(Z)-dienoic acid (6-(S))

(1) The reaction was carried out substantially in the manner as in Example 1 (3), but using (R)-4-dodecanolide, to afford 6-1 as a mixture of anomers.

¹H NMR (CDCl₃, 300 MHz) δ ppm: 0.88 (t, J=7.2 Hz, 3H), 1.22-2.20 (m, 18H), 2.76 (d, J=3.3 Hz, 0.41H), 2.81 (d, J=3.3 Hz, 0.59H), 3.93-4.02 (m, 0.41H), 4.15-4.23 (m, 0.59H), 5.45-5.47 (m, 0.41H), 5.53-5.56 (m, 0.59H).

(2) The reaction was carried out substantially in the manner as in Example 1 (4), but using the compound 6-1 obtained in the above (1) to afford 6-2-(R).

¹H NMR (CDCl₃, 400 MHz) δ ppm: 0.88 (t, J=6.7 Hz, 3H), 1.20-1.62 (m, 16H), 1.70 (quint, J=7.2 Hz, 2H), 2.07-2.26 (m, 4H), 2.33 (t, J=7.6 Hz, 2H), 2.80 (t, J=6.0 Hz, 2H), 3.57-0.64 (m, 1H), 3.67 (s, 3H), 5.33-5.44 (m, 4H); ¹³C NMR (CDCl₃, 75.3 MHz) δ ppm: 14.31, 22.87, 23.80, 24.98, 25.81, 25.89, 26.76, 29.48, 29.80, 29.91, 32.09, 33.63, 37.39, 37.78, 51.73, 71.75, 128.48, 128.98, 129.25, 129.96, 174.38.

(3) The reaction was carried out substantially in the manner as in Example 1 (5), but using the compound 6-2-(R), obtained in the above (2), to afford 6-3.

¹H NMR (CDCl₃, 400 MHz) δ ppm: 0.86 (t, J=6.6 Hz, 3H), 1.20-1.42 (m, 12H), 2.00-2.07 (m, 6H), 2.03 (quart, J=6.9 Hz, 2H), 2.27 (t, J=7.8 Hz, 2H), 2.72 (t, J=5.7 Hz, 2H), 3.66 (s, 3H), 5.11-5.19 (m, 1H), 5.27-5.44 (m, 4H), 7.44 (t, J=7.2 Hz, 2H), 7.56 (t, J=7.5 Hz, 1H), 8.06 (d, J=7.2 Hz, 2H)

(4) The reaction was carried out substantially in the manner as in Example 1 (6), but using the compound 6-3, obtained in the above (3), to afford 6-2-(S) (40%) and 6-3 was recovered (56%).

¹H NMR of 6-2-(S) was identical with that of 6-2-(R).

(5) The reaction was carried out substantially in the manner as in Example 1 (7), but using the compound 6-2-(S), obtained in the above (4), to afford the title compound 6-(S).

¹H NMR (CDCl₃, 400 MHz) δ ppm: 0.88 (t, J=7.2 Hz, 3H), 1.20-1.60 (m, 16H), 1.70 (quint, J=7.6 Hz, 2H), 2.06-2.18 (m, 3H), 2.21-2.32 (m, 1H), 2.36 (t, J=7.2 Hz, 2H), 2.74-2.88 (m, 2H), 3.62-3.65 (m, 1H), 5.32-5.48 (m, 4H).

### Example 12

### Synthesis of 12(R)-Hydroxyeicosa-5(Z),8(Z)-dienoic acid (6-(R))

The reaction was carried out substantially in the manner as in Example 1 (7), but using the compound 6-2-(R), obtained in Example 11 (2), to afford the title compound 6-(R).

¹H NMR of 6-(R) was identical with that of 6-(S).

### Example 13

### Synthesis of 12(R)-Hydroxy-eicosa-5(Z),14(Z)-dienoic acid (7-(R))

(1) The reaction was carried out substantially in the manner as in Example 5 (1), but using 10-(t-butyldiphenysilanyloxy)dec-5(Z)-en-1-ol [James A. Marshall and Xiao-jun Wang J. Or Chem., 1992, 57, 3387.], to afford 7-1.

¹H NMR (CDCl₃, 300 MHz) δ ppm: 1.05 (s, 9h), 1.39-1.59 (m, 6H), 1.85 (quint, J+8.1 Hz, 2H), 2.02 (quint, J=6.0 Hz, 4H), 3.39 (t, J=6.6 Hz, 2H), 3.66 (t, J=6.3 Hz, 2H), 5.29-5.42 (m, 2H), 7.34-7.43 (m, 6H), 7.65-7.68 (m, 4H).

(2) t-BuLi (1.7 M in pentane, 1.83 mL, 3.12 mmol) was added dropwise over 5 minutes to a stirring, −78° C. solution of the compound obtained in the above (1), 7-1 (0.741 g, 1.56 mmol), in dry ether (10 mL). The reaction mixture was slowly warmed to −20° C. over 30 min, then recooled to −78° C. and transferred via cannula to a stirring, −78° C. suspension of CuCN (0.066 g, 0.078 mmol) in dry ether (2 mL). The mixture was slowly warned to −30° C. over 1 h, then recooled to −78° C. (S)-1,2-Epoxydec-4(Z)-ene [Stephen W. Russel and Henk J. J. Pabon J. Chem Soc., Perkin Trans I, 1982, 545.] dissolved in ether (3 mL) was added dropwise to the copper reagent at −78° C. and the resultant mixture was stirred at that temperature for another 2 h. Saturated aqueous NH₄Cl solution (5 mL) followed by 10% aqueous NH₄OH solution (2 mL) were added, the mixture was then brought to room temperature, and extracted with ether (10 mL×3). The combined ethereal extracts were dried over anhydrous MgSO₄, concentrated under vacuum, and the residue was subjected to silica gel column chromatography (AcOEt/hexane, 1:15) to yield 0.077 g (ca. 27%) of 7-2, which was used to the next reaction without further purification.

(3) Benzoyl chloride (0.138 g, 0.985 mmol) was added to a stirring, room temperature solution of 7-2 (0.180 g, 0.328 mmol), Et₃N (0.199 g, 1.968 mmol), and 4-(dimethylamino)pyridine (DMAP) (0.020 g, 0.164 mmol) in dry CH₂Cl₂ (5 mL). After 36 h, the solvent was evaporated and the residue was subjected to silica gel column chromatography to yield 0.171 g (80%) of 7-3.

¹H NMR (CDCl₃, 400 MHz) δ ppm: 0.86 (t, J=6.8 Hz, 3H), 1.05 (s, 9H), 1.20-1.46 (m, 14H), 1.52-1.60 (m, 2H), 1.62-1.76 (m, 2H), 1.94-2.08 (m, 6H), 2.36-2.52 (m, 2H), 3.65 (t, J+6.4 Hz, 2H), 5.14 (quint, J=6.2 Hz, 1H), 5.28-5.52 (m, 4H), 7.36-7.45 (m, 8H), 7.55 (t, J=8.0 Hz, 1H), 7.67 (d, J=8.0 Hz, 4H), 8.04 (d, J=6.8 H, 2H).

(4) n-Bu₄NF (1.04 mL, 1.0 M solution in THF, 1.04 mmol) was added to a stirring, room temperature solution of compound 7-3 (0.227 g, 0.348 mmol) in dry THF (8 mL). After stirring for 12 h, the solvent was evaporated under vacuum and the residue was purified by silica gel column chromatography using AcOEt/hexane (1:9) as eluant to give 7-4 (0.142 g, 98%).

¹H NMR (CDCl₃, 400 MHz) δ ppm: 0.86 (t, J=6.9 Hz, 3H), 1.24-2.10 (m, 24H), 2,34-2.52 (m, 2H), 3.60-3.66 (m, 2H), 5.08-5.38 (quint, J=6.0 Hz, 1H), 5.32-5.54 (m, 4H), 7.41-7.46 (m, 2H), 7.52-7.58 (m, 1H), 8.02-8.06 (m, 2H).

(5) Pyridinium dichromate (PDC) (0.645 g, 1.715 mmol) was added to a stirring, room temperature solution of 7-4 (0.142 g, 0.343 mmol) in dry N,N-dimethylformamide (DMF) (5 mL). After 15 h, the reaction mixture was diluted with water (10 mL) and extracted with AcOEt (5 mL×3). The combined organic extracts were washed with water, brine, dried over MgSO₄, and the solvent was evaporated under vacuum. The residue was redissolved in ether/methanol (1:1, 5 mL), cooled to 0° C., and treated with excess CH₂N₂ in ether. Evaporation of all volatiles in vacuo and silica gel chromatography of the residue using AcOEt/hexane (1:20) as eluant gave 7-5-(R) (0.080 g, 53%).

¹H NMR (CDCl₃, 400 MHz) δ ppm: 0.86 (t, J=6.8 Hz, 3H), 1.20-1.44 (m, 12H), 1.62-1.75 (m, 4H), 1.94-2.09 (m, 6H), 2.29 (t, J=7.6 Hz, 2H), 2.35-2,51 (m, 2H), 3.66 (s, 3H), 5.14 (quint, J=6.4 Hz, 1H), 5.27-5.52 (m, 4H), 7.43 (t, J=7.6 Hz, 2H), 7.55 (t, J=7.2 Hz, 1H), 8.04 (d, J=7.2 Hz, 2H).

(6) The reaction was carried out substantially in the manner as in Example 1 (6), but using the compound 7-5-(R), obtained in the above (5), to afford 7-6-(R).

¹H NMR (CDCl₃, 400 MHz) δ ppm: 0.89 (t, J=6.8 Hz, 3H), 1.24-1.57 (m, 14H), 1.68 (quint, J=7.2 Hz, 2H), 1.99-2.10 (m, 6H), 2.21 (t, J=7.0 Hz, 2H), 2.32 (t, J=7.6 Hz, 2H), 3.57-3.65 (m, 1H), 3.67 (s, 3H), 5.28-5.44 (m, 3H), 5.53-5.61 (m, 1H); ¹³C NMR (CDCl₃, 75.3 MHz) δ ppm: 14.23, 22.73, 25.05, 25.80, 26.71, 27.29, 27.56, 29.46, 29.52, 29.78, 31.31, 33.63, 35.55, 36.97, 51.65, 71.62, 125.29, 128.61, 131.19, 133.66, 174.35.

(7) The reaction was carried out substantially in the manner as in Example 1 (7), but using the compound 7-6-(R), obtained in the above, to afford the title compound 7-(R).

¹H NMR (CDCl₃, 400 MHz) δ ppm: 0.89 (t, J=7.2 Hz, 3H), 1.24-1.52 (m, 14H), 1.69 (quint, J=7.2 Hz, 2H), 1.9-2.14 (m, 6H), 2.22 (t, J=7.0 Hz, 2H), 2.36 (t, J=7.2 Hz, 2H), 3.60-3.68 (m, 1H), 5.28-5.46 (m, 3H), 5.52-5.62 (m, 1H).

### Example 14

### Synthesis of 12(S)-Hydroxyeicosa-5,(Z)14(Z)-dienoic acid (7-(S))

(1) The reaction was carried out substantially in the manner as in Example 1 (5), but using the compound 7-6-(R), obtained in Example 13 (6), to afford methyl 12 (S)-benzoyloxyeicosa-5(Z), 14(Z)-dienoate.

¹H NMR of the compound thus obtained was identical with that of 7-5-(R).

(2) The reaction was carried out substantially in the manner as in Example 1 (6), but using the compound obtained in the above (1), to afford methyl 12(S)-hydroxyeicosa-5(Z), 14(Z)-dienoate.

¹H NMR of the compound thus obtained was identical with that of 7-6-(R).

(3) The reaction was carried out substantially in the manner as in Example 1 (7), but using the compound obtained in the above (2), to afford title compound 7-(S).

¹H NMR of the compound thus obtained was identical with that of 7-(R).

### Example 15

### Measurements of Agonistic and Antagonistic Activity of 12(R)-HETrE Analogs

Incubation of microvessel endothelial (RLMVE) cells with 12(R)-HETrE at concentration of 0.1 nM resulted in a rapid induction of VEGF expression. 12(R)-HETrE increased VEGF mRNA levels in a time-dependent manner. A 5-fold increase over the control levels was observed 45 min after addition of 12(R)-HETrE. These levels gradually declined to the control levels by 48 h. Incubation of cells with cycloheximide did not affect 12(R)-HETrE-induced VEGF mRNA, suggesting that the induction does not require de novo protein synthesis. In contrast, addition of actinomycin D abolished 12(R)-HETrE-induced VEGF expression indicating that this effect required de novo RNA synthesis. Measurements of the half-life of VEGF mRNA in the presence of 12(R)-HETrE suggested that both transcriptional activation and mRNA stabilization accounted for the increase in VEGF mRNA induced by 12(R)-HETrE. 12(R)-HETrE increased VEGF mRNA in a concentration-dependent manner with 0.1 nM having the maximal effect.

Human VEGF cDNA was used as a probe for slot blot analysis to measure increases in VEGF mRNA following incubation of RLMVE cells with 12(R)-HETrE and 12-HETrE analogs. To measure agonistic activity compounds were added to the incubation medium at concentration of 0.1, 1, and 10 nM and their effect on VEGF mRNA levels after 45 min incubation was measured in comparison to that of 12(R)-HETrE at the same concentration. To measure antagonistic activity, compounds at the above concentrations were added with 0.1 nM 12(R)-HETrE and 45 min later RNA extracted samples were processed for slot blot analysis. Densitometry analysis indicated that 1-a-(S) exhibited significant agonistic activity, increasing VEGF mRNA by 5-fold at 1 nM.

We claim:

1. A compound of the formula: 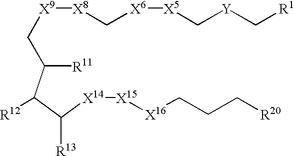
wherein X⁵, X⁶, X⁸, X⁹, X¹⁴, X¹⁵, and X¹⁶ are independently selected from the group consisting of —CH₂—, —CH—, and —C—, and wherein X⁵═X⁶, X⁸═X⁹, and X¹⁴═X¹⁵ or X¹⁵═X¹⁶;
R¹ is selected from the group consisting of —COOH, —CH₃, and —C(O)NHSO₂Z, wherein Z is selected from the group consisting of methyl, paraiodobenzene, —COOH, parabenzylamine, propylamine, and COOR²¹, and wherein R²¹ is alkyl having from 1 to 6 carbons;
R¹¹, R¹², and R¹³ are independently selected from the group consisting of —H, —OH, and —OCH₃;
R²⁰ is —CH₃ or —COOH; and
Y is selected from the group consisting of —CH₂—, —O—, —N—, and —S—;
with the proviso that when X⁵, X⁸, and X¹⁴ are —CH—, Y is —CH₂—, R¹ is —COOH, R¹¹ and R¹³ are both H, and R²⁰ is —CH₃, R¹² is not OH:

2. A compound according to claim 1 wherein at least one of R¹¹, R¹² and R¹³ define a stereocenter with an (R) configuration.

3. A compound according to claim 1 wherein at least one of R¹¹, R¹² and R¹³ define a stereocenter with an (S) configuration.

4. A compound according to claim 1 wherein X⁸ and X¹⁴ are each —CH—.

5. A compound according to claim 1 wherein R¹ is —COOH and R²⁰ is CH₃.

6. A compound according to claim 1 wherein only one of R¹¹, R¹², and R¹³is —OH.

7. A compound according to claim 1 wherein Y is —CH₂—.

8. A compound of the formula: 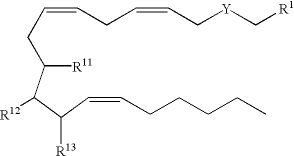
wherein R¹ is selected from the group consisting of —COOH, —CH₃, and —C(O)NHSO₂Z, wherein Z is selected from the group consisting of methyl, paraiodobenzene, —COOH, parabenzylamine, propylamine, and COOR²¹, and wherein R²¹ is alkyl having from 1 to 6 carbons;
R¹¹, R¹² and R¹³ are independently selected from the group consisting of —H, —OH, and —OCH₃; and
Y is selected from the group consisting of —CH₂—, —S—, —N—, and —O—;
with the proviso that when Y is —CH₂—, R¹ is COOH, and R¹¹ and R¹³ are each —H, R¹² is not —OH.

9. A compound according to claim 8 wherein at least one of R¹¹, R¹² and R¹³ define a stereocenter with an (R) configuration.

10. A compound according to claim 8 wherein at least one of R¹¹, R¹² and R¹³ define a stereocenter with an (S) configuration.

11. A compound according to claim 8 selected from the group consisting of:

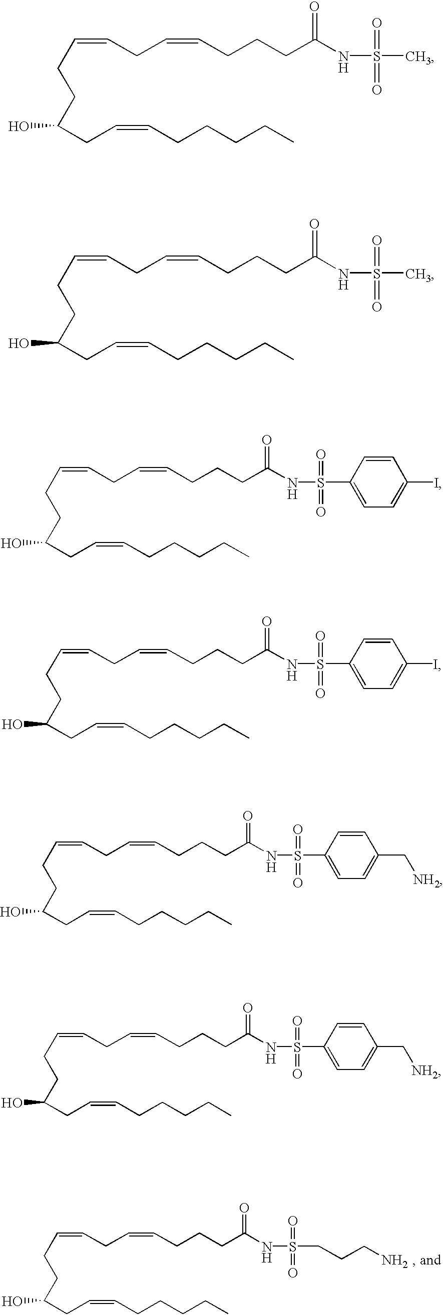

12. A compound according to claim 8 selected from the group consisting of:

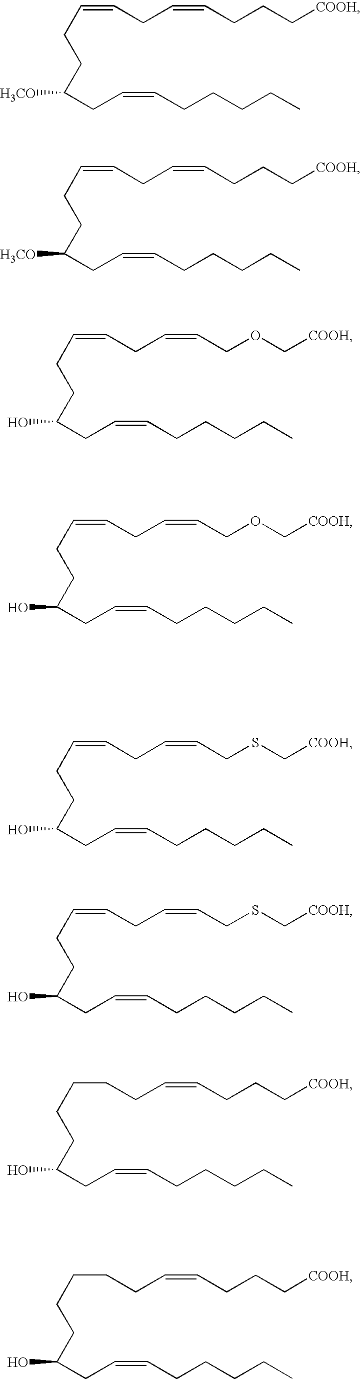

13. A compound of the formula: 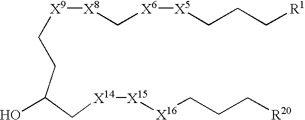
wherein R¹ and R²⁰ are independently selected from the group consisting of —CH₃ and —COOH; and
X⁵, X⁶, X⁸, X⁹, X¹⁴, X¹⁵, and X¹⁶ are independently selected from the group consisting of —CH₂—, —CH—, and —C—, and wherein X⁵═X⁶, X⁸═X⁹, and X¹⁴═X¹⁵ or X¹⁵═X¹⁶;
with the proviso that when R¹ is COOH, R²⁰ is —CH₃, and X⁵ and X⁸ are each —CH—, X¹⁴ is not —CH—.

14. A compound according to claim 13 wherein the hydroxy group at the 12 position defines a stereocenter with an (R) configuration.

15. A compound according to claim 13 wherein the hydroxy group at the 12 position defines a stereocenter with an (S) configuration.

16. A compound according to claim 13 selected from the group consisting of:

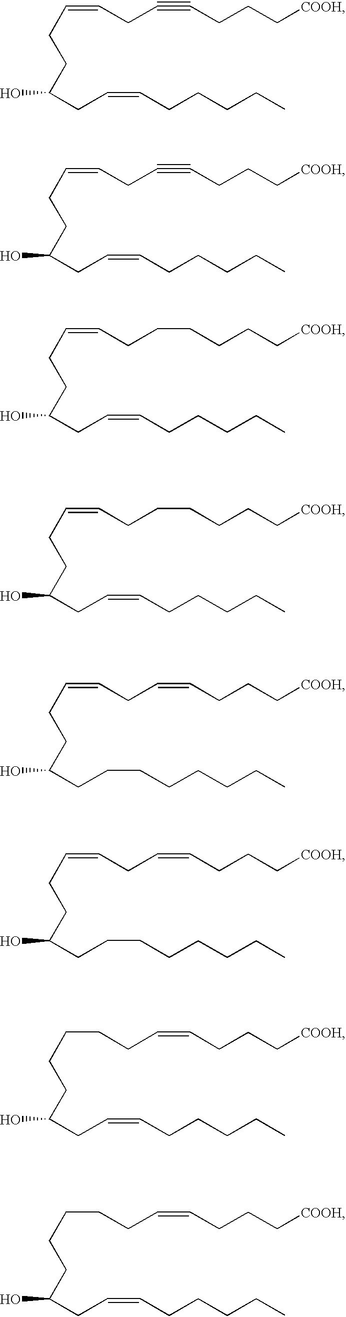

17. A compound of the formula: 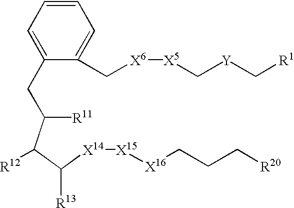
wherein X⁵, X⁶, X¹⁴, X¹⁵, and X¹⁶ are independently selected from the group consisting of —CH₂—, —CH—, —C—, —N—, —O—, and —S—;
R¹ is selected from the group consisting of —COOH, —CH₃, and —C(O)NHSO₂Z, wherein Z is selected from the group consisting of methyl, paraiodobenzene, —COOH, parabenzylamine, propylamine, and COOR²¹, and wherein R²¹ is alkyl having from 1 to 6 carbons;
R¹¹, R¹², and R¹³ are independently selected from the group consisting of —H, —OH, and —OCH₃;
R²⁰ is —CH₃ or —COOH; and
Y is selected from the group consisting of —CH₂—, —O—, —N—, and —S—.

18. A compound of the formula: 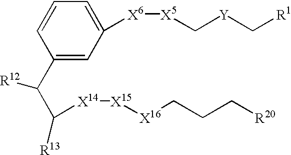
wherein X⁵, X⁶, X¹⁴, X¹⁵, and X¹⁶ are independently selected from the group consisting of —CH₂—, —CH—, —C—, —N—, —O—, and —S—;
R¹ is selected from the group consisting of —COOH, —CH₃, and —C(O)NHSO₂Z, wherein Z is selected from the group consisting of methyl, paraiodobenzene, —COOH, parabenzylamine, propylamine, and COOR²¹, and wherein R²¹ is alkyl having from 1 to 6 carbons;
R¹² and R¹³ are independently selected from the group consisting of —H, —OH, and —OCH₃;
R²⁰ is —CH₃ or —COOH; and
Y is selected from the group consisting of —CH₂—, —O—, —N—, and —S—.

19. A compound of the formula: 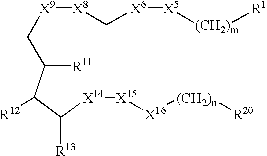
wherein X⁵, X⁶, X⁸, X⁹, X¹⁴, X¹⁵, and X¹⁶ are independently selected from the group consisting of —CH₂—, —CH—, —C—, —N—, —O—, and —S—;
R¹ is selected from the group consisting of —COOH, —CH₃, and —C(O)NHSO₂Z, wherein Z is selected from the group consisting of methyl, paraiodobenzene, —COOH, parabenzylamine, propylamine, and COOR²¹, and wherein R²¹ is alkyl having from 1 to 6 carbons;
R¹¹, R¹², and R¹³ are independently selected from the group consisting of —H. —OH, and —OCH₃;
R²⁰ is —CH₃ or —COOH;
m is an integer ranging from 0 to 6; and
n is an integer ranging from 0 to 6; with the proviso that when X⁵, X⁸, and X¹⁴ are —CH—, m is 3, n is 3, R¹ is COOH, and R¹¹ and R¹³ are both H, R¹² is not OH.

20. A method of inhibiting chemotaxis in a cell comprising:
administering a 12-HETrE antagonist to the cell in an amount effective to inhibit chemotaxis in the cell.

21. The method of claim 20, wherein the cell is a neutrophil.

22. A method for treating or preventing inflammation in a subject comprising:
administering to the subject having an adverse medical condition characterized by inflammation, a 12-HETrE antagonist in an amount effective to inhibit the inflammation.

23. The method of claim 22 wherein the adverse medical condition is a skin inflammatory condition.

24. The method of claim 23 wherein the skin inflammatory condition is hypersensitization.

25. The method of claim 23 wherein the skin inflammatory condition is psoriasis.

26. The method of claim 22 wherein the adverse medical condition is a corneal inflammatory condition.

27. The method of claim 22 wherein the inflammation is mediated by neutrophils.

28. The method of claim 22 wherein the inflammation is mediated by leukocytes.

29. The method of claim 22 wherein the inflammation is mediated by T cells.

30. The method of claim 22 wherein the inflammation comprises at least one of vasodilation, an increase in membrane permeability, early neutrophil chemotaxis, and late angiogenesis.

31. The method of claim 22 wherein the 12-HETrE antagonist is administered to the subject orally, intravenously, transdermally, intraparenterally, subcutaneously, intramuscularly, or intracavitally.

32. The method of claim 22 wherein the 12-HETrE antagonist is:

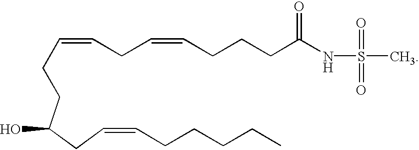

33. The method of claim 22 wherein the 12-HETrE antagonist is:

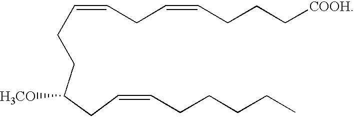

34. The method of claim 22 wherein the 12-HETrE antagonist is:

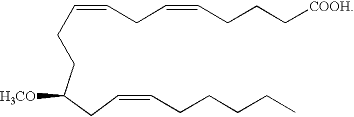

35. The method of claim 22 wherein the 12-HETrE antagonist is 12(S)-hydroxy-5,8,14-eicosatrienoic acid.

36. The method of claim 22 wherein the 12-HETrE antagonist is:

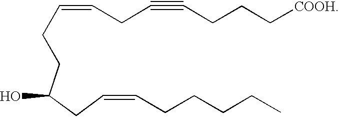

37. The method of claim 22 wherein the 12-HETrE antagonist is:

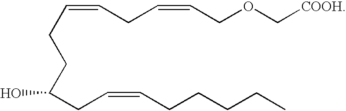

38. The method of claim 22 wherein the 12-HETrE antagonist is:

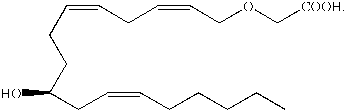

39. A method of treating a subject having an ocular condition comprising:
administering to the subject a 12-HETrE antagonist in an amount effective to treat the ocular condition.

40. The method of claim 39 wherein the ocular condition is selected from the group consisting of corneal angiogenesis, corneal inflammation, corneal transplantation, injury due to contact lens wear, trachoma, infectious conditions, retinal neovascularization, choroidal neovascularization, retinopathy, and age-related macular degeneration.

41. The method of claim 40 wherein the retinopathy is selected from the group consisting of retinopathy of prematurity and diabetic retinopathy.

42. The method of claim 39 wherein the ocular condition is caused by a viral infection or a bacterial infection.

43. The method of claim 39 wherein the ocular condition is an injury due to prolonged contact lens wear.

44. The method of claim 39 wherein the ocular condition is characterized by neovascularization.

45. The method of claim 39 wherein the 12-HETrE antagonist is administered to the subject orally, intravenously, transdermally, intraparenterally, subcutaneously, intramuscularly, or intracavitally.

46. The method of claim 39 wherein the 12-HETrE antagonist is 12(S)-hydroxy-5,8,14-eicosatrienoic acid.

47. The method of claim 39 wherein the 12-HETrE antagonist is:

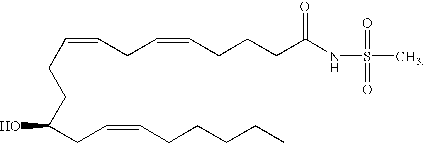

48. The method of claim 39 wherein the 12-HETrE antagonist is:

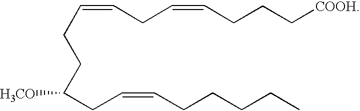

49. The method of claim 39 wherein the 12-HETrE antagonist is:

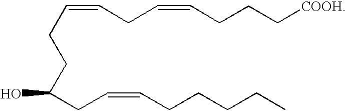

50. The method of claim 39 wherein the 12-HETrE antagonist is 12(S)-hydroxy-5,8,14-eicosatrienoic acid.

51. The method of claim 39 wherein the 12-HETrE antagonist is:

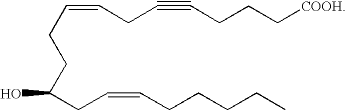

52. The method of claim 39 wherein the 12-HETrE antagonist is:

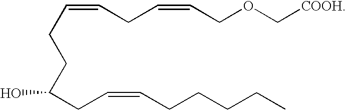

53. The method of claim 39 wherein the 12-HETrE antagonist is:

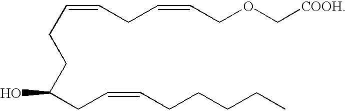

54. A method for treating a cardiovascular disorder in a subject comprising:
administering to the subject having the cardiovascular disorder a 12-HETrE agonist in an amount effective to treat the cardiovascular disorder.

55. The method of claim 54 wherein the cardiovascular disorder is an ischemic condition.

56. The method of claim 55 wherein the ischemic condition is selected from the group consisting of stroke, myocardial infarction, and coronary artery disease.

57. The method of claim 54 wherein the 12-HETrE agonist is administered to the subject orally, intravenously, transdermally, intraparenterally, subcutaneously, intramuscularly, or intracavitally.

58. The method of claim 54 wherein the cardiovascular disorder is caused by at least one of diabetes and aging.

59. The method of claim 54, further comprising administering an angiogenic factor.

60. The method of claim 57 wherein the angiogenic factor is selected from the group consisting of angiogenin, angiopoietin-1, Del-1, acidic fibroblast growth factor, basic fibroblast growth factor, follistatin, granulocyte colony-stimulating factor, hepatocyte growth factor, scatter factor, interleukin-8, leptin, midkine, placental growth factor, platelet-derived endothelial cell growth factor, platelet-derived growth factor-BB, pleiotrophin, proliferin, transforming growth factor-alpha, transforming growth factor-beta, tumor necrosis factor-alpha, vascular endothelial growth factor, and vascular permeability factor.

61. The method of claim 56 wherein the angiogenic factor is selected from the group consisting of acidic fibroblast growth factor, basic fibroblast growth factor, and vascular endothelial growth factor.

62. The method of claim 54 wherein the 12-HETrE agonist is:

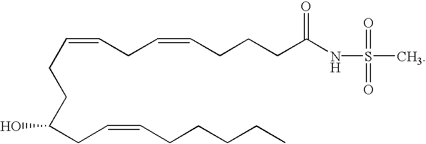

63. The method of claim 54 wherein the 12-HETrE agonist is:

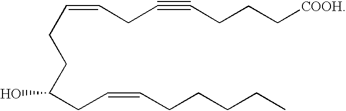

64. The method of claim 54 wherein the 12-HETrE agonist is:

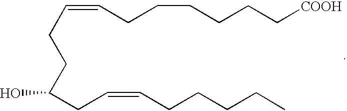

65. The method of claim 54 wherein the 12-HETrE agonist is:

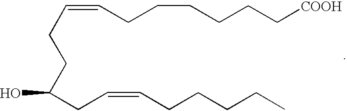

66. The method of claim 54 wherein the 12-HETrE agonist is:

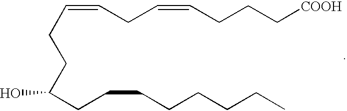

67. The method of claim 54 wherein the 12-HETrE agonist is:

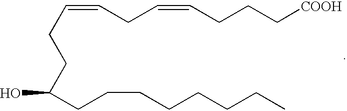

68. The method of claim 54 wherein the 12-HETrE agonist:

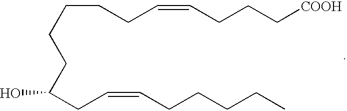

69. The method of claim 54 wherein the 12-HETrE agonist is:

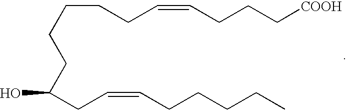

70. The method of claim 54 wherein the 12-HETrE agonist is:

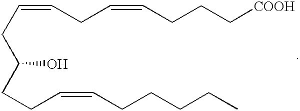

71. The method of claim 54 wherein the 12-HETrE agonist is:

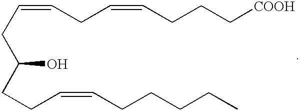

72. A method for treating cancer in a subject comprising:
administering to the subject in need of such treatment, a 12-HETrE antagonist in an amount effective to treat the cancer.

73. A method for inhibiting cell growth in a tumor comprising:
administering a 12-HETrE antagonist to the tumor in an effective amount to inhibit cell growth.
